(19) United States
(12) Patent Application Publication
(10) Pub. No.: US 2009/0220516 A1
(43) Pub. Date: Sep. 3, 2009

(54) Neuroprotection of retinal ganglion cells

(76) Inventors: Alan Laties, Philadelphia, PA (US); Claire Mitchell, Philadelphia, PA (US)

Correspondence Address: Pearl Cohen Zedek Latzer, LLP, 1500 Broadway, 12th Floor, New York, NY, 10036, US

(21) Appl. No.: 11/990,703

(22) PCT Filed: Jun. 21, 2006
(86) PCT No.: PCT/US06/24063

§ 371 Date: Apr. 22, 2009

Related U.S. Application Data
(60) Provisional application No. 60/692,657, filed on Jun. 22, 2005.
(60) Provisional application No. 60/718,721, filed on Sep. 21, 2005.

(51) Int. Cl. A61K 39/395; A61K 38/45; A61K 31/70; A61K 31/66; A61K 31/13

(52) U.S. Cl. 424/141.1; 424/94.5; 514/46; 514/114; 514/662

(57) ABSTRACT

This invention relates to the neuroprotection of the optic nerve and the treatment of glaucoma, more specifically, the invention is directed to a method of preventing, inhibiting, decreasing incidence and suppressing death in ganglion cells by manipulating the P2X₇ and A₃ receptors on ganglion cells, by reducing levels of ATP released into the extracellular space of the retina and enhancing the conversion of released extracellular ATP into adenosine.

### CROSS REFERENCE TO RELATED APPLICATIONS

This application is a PCT International Application claiming priority from U.S. Provisional Patent Application No. 60/692,657, filed 22 Jun. 2005 and U.S. Provisional Patent Application No. 60/718,721, filed 21 Sep. 2005, both which are hereby incorporated by reference in their entirety

### FIELD OF INVENTION

The invention is directed to compositions and methods for the neuroprotection of the optic nerve and the treatment of glaucoma, as well as chronic glaucoma. Specifically, the invention is directed to methods and compositions for preventing, inhibiting, decreasing incidence and suppressing death of retinal ganglion cells by manipulating the P2X₇ and A₃ receptors on ganglion cells, by reducing the level of excess extracellular ATP and by converting excess ATP into adenosine.

### BACKGROUND OF THE INVENTION

Glaucoma is the second leading cause of blindness in the world, The disease is characterized by a death of ganglion cells in the retina (RGCs). As ganglion cell axons form the optic nerve and bring visual information to the brain, their death directly affects visual performance. The best characterized forms of glaucoma are associated with elevations in intraocular pressure mainly due to a decrease in the rates of aqueous humor drainage through the aqueous drainage channels. Current pharmacologic treatment for glaucoma is confined to reducing pressure by increasing the rates of aqueous humor drainage, or decreasing the production of aqueous humor to match the reduced outflow. However, pressure lowering is only partially effective. Ganglion cell loss can continue after pressure has been brought under control. Consequently, a need exists for the neuroprotection of retinal ganglion cells. The ability to preserve ganglion cells in glaucoma is presently hampered by the inability to explain why elevated pressure leads to cell loss in the first place. It is suggested that elevated pressure produces a distention of the lamina cribosa, a latticed scaffolding structure supporting the optic nerve fibers as they leave the eye. Under high pressures, distention is sufficient to inhibit the transport of neurotrophic factors from the brain to the ganglion cell body in the retina. However, glaucoma pathology can occur even in the absence of elevated pressure, and ganglion cell loss can continue long after an elevated pressure has been brought under control. Several observations indicate that cytotoxic damage is initiated in the cell bodies residing within the retina. Over-stimulation of the NMDA glutamate receptor leads to an elevation of intracellular Ca²⁺ (Ca²⁺;) and activation of apoptotic cell death. The NMDA antagonist memantine and various apoptosis inhibitors can reduce the rate of NMDA-triggered cell death. Elevation of Ca²⁺i is thought to be an essential early step in the cell body-mediated death, and this Ca²⁺i increase may induce apoptotic loss by activation of endonucleases and proteases.

### SUMMARY OF THE INVENTION

In one embodiment, the invention provides a method of reducing the release of cytotoxic ATP from a retinal cell in response to elevated intraocular pressure, comprising contacting said cell with an inhibitor of ATP release, thereby decreasing the release of excess ATP into the retina in response to elevated pressure.

In another embodiment, the invention provides a method for enhancing the conversion of ATP into adenosine in a retinal ganglion cell, comprising contacting said cell with an ecto-nucleotidase agonist and removing ATP thereby producing adenosine.

In another embodiment, the invention provides a method for the neuroprotection of retinal ganglion cells comprising stimulating an adenosine receptor on the retinal ganglion cells, thereby preventing an excess Ca²⁺influx and death of retinal ganglion cells.

In one embodiment, the invention provides a composition comprising at least two of a P2X₇ receptor antagonist, an adenosine A₃ receptor agonist, an adenosine A₁ receptor agonist, an agent capable of blocking the release of excessive ATP in response to elevated intraocular pressure, an ecto-nucleotidase agonist to convert extracellular ATP into adenosine, a Ca²⁺ chelating agent, an NMDA receptor antagonist.

In another embodiment, the invention provides a method for inhibiting or suppressing the reduction in number of retinal ganglion cells in a subject, comprising administering to said subject an effective amount of a P2X₇ antagonist, thereby preventing the stimulation of P2X₇ receptors leading to death of ganglion cells and a reduction in their numbers.

In one embodiment, the invention provides a method of treating a pathological condition in a subject resulting from a reduction in number of retinal ganglion cells, comprising administering to said subject a composition comprising at least two of a P2X₇ receptor antagonist, an adenosine A₃ receptor agonist, an adenosine A₁ receptor agonist, an agent capable of blocking the release of excessive ATP in response to elevated intraocular pressure, an ecto-nucleotidase agonist to convert extracellular ATP into adenosine, a Ca²⁺ chelating agent, an NMDA receptor antagonist, thereby reducing the stimulation of the P2X₇ receptors leading to death of ganglion cells, a reduction in their number thereby resulting in loss of function of said retinal ganglion cells.

### BRIEF DESCRIPTION OF THE DRAWINGS

FIG. 1. shows the ability of P2X₇ antagonist KN04 to block the effects of BzATP on ganglion cells.

FIG. 2. In ganglion cells from mixed retinal cultures, NMDA receptor antagonists reduce Ca²⁺ elevation triggered by P2X₇ receptor activation. (A) Application of 50 μM BzATP (Bz) for 15 sec led to a large increase in Ca²⁺ levels that returned to normal after removal of BzATP. Duration of drug application is indicted by lines over the trace. Reapplication after 6 min wash led to an elevation similar to the first, with multiple responses evident. (B) Application of 10 μM MK-801 reduced the Ca²⁺ elevation triggered by 50 mM BzATP. A substantial block of the BzATP response was also found with 100 μM APV (C) and 30 μM memantine (D). E. Summary of the effects of NMDA receptor blockers on response to BzATP in to ganglion cells from mixed retinal neurons. Bars represent the mean±SEM; Bz is the mean rise to applications 1 and 3 (BzATP alone), while the value for each drug is the mean increase to applications 2 and 4. All values were normalized to the increase detected in the first application of BzATP alone. 10 μM MK-801, N=5, * p=0.019; 300 μM APV, N=5, ** p=0.033; 30 μM memantine, N=13, *** p=0.002; All unpaired Students t-test.

FIG. 3. In isolated retinal ganglion cells, NMDA antagonists reduce Ca²⁺ elevation triggered by BzATP. While repeated 15 sec applications of 50 μM BzATP (Bz) led to reproducible elevations in Ca²⁺, (A) the response was reduced by addition of 10 μM MK-801 (B), 100 μM APV (C) or 30 μM memantine (D). (E) Summary of results, MK-801; N=13* p<0.0001; APV, N=10** p=0.002; memantine, N=10, *** p=0.015. Students t-test.

FIG. 4 shows stimulation of the P2X₇ receptor triggers glutamate release from ganglion cells. BzATP (50 μM) led to large, reversible and repeatable release of glutamate from isolated ganglion cells. Cells were perfused with the glutamate dehydrogenase mixture and fluorescence determined at 0.5 Hz. Application of BzATP for 15 sec. led to a rapid increase in fluorescence. Cells were washed with enzyme-free solution for 4 min between trials. A fixed number of ganglion cells were present in the field in this particular experiment. F360 is the fluorescence excited at 360 nm, an index of NADH production from released glutamate.

FIG. 5. Immunological and functional co-localization of P2X₇ and NMDA receptors. (A). Isolated retinal ganglion cells stained for the P2X₇ receptor with the antibody to AAs 136-152. Punctate staining is detectable in some regions, with lighter stain over the nucleus (B) The same cell co-stained for the NMDA receptor. The staining pattern is similar, with punctate focus also visible. (C) DIC image of the cell. The surface over the nucleus appears relatively smooth. (D). Overlay of images A-C. While co-localization of P2X₇ and NMDA receptors is apparent in some locations, particulate clumps of either receptor are also observed alone. Scale bar=10 μm. (E) The proportion of cells staining for NMDAR or P2X₇R. Bars represent the mean±SEM with data including data from P2X₇R antibodies AA 136-152 and AA 576-595. The quantification of demonstrates that the majority of cells contained NMDA receptors (white) and P2X₇ receptors (black), N=40. (F) Functional analysis of receptor frequency. Bars indicate the percentage of cells responding to agonists with an elevation in Ca²⁺. Cells were exposed to either 10 μM glutamate with 10 μM glycine (white) or 50 mM BzATP (black) for 15-30 sec. The effect of the first application only was counted, with an increase above threshold indicating a response. N=21 for glutamate/glycine and 27 for BzATP.

FIG. 6 shows NMDA antagonists reduce lethal effects of BzATP. (A) While incubation with 50 μM BzATP for 24 hrs reduced the number of surviving retinal ganglion cells compared to that in control solution, this loss was prevented by 10 μM MK-801 (*—diff from BzATP alone, Dunnett's test, N=9). (B) The antagonist APV (100 mM) also increased cell survival (*—diff from BzATP alone, Dunnett's test, N=15). (C) At 100 μM, memantine was also neuroprotective, raising the number of surviving cells considerably above that found in BzATP alone (*—diff from BzATP alone, Dunn's test on ranks due to enhanced variation, memantine not different from control, N=18).

FIG. 7 shows that adenosine dampens the rise in Ca²⁺ triggered by BzATP. (A) Application of 50 μM BzATP for 15 sec lead to large, reversible increases in Ca²⁺. Repeated application of BzATP produced multiple elevations in Ca²⁺ that were roughly uniform in size. Experiments were performed a single ganglion cell labeled with fura-2 present in mixed retinal cultures in the absence of Mg²⁺. (B) Adenosine prevented the rise in Ca²⁺ triggered by BzATP. Application of adenosine greatly attenuated the response to 50 μM BzATP. (C) Quantification of the adenosine block of the rise Ca²⁺ rise. Bars represent the mean±SE of the rise in Ca²⁺ triggered by 50 μM BzATP with and without adenosine (n=8). To control for any small decrease between responses, each “BzATP” value is the mean response to the 1st and 3rd application (BzATP alone) while (Ado) is the mean of the 2nd and 4th applications. * p=0.0013, paired Student's t-test.

FIG. 8 shows adenosine is a neuroprotective agent. A. Adenosine (Ado, 300 μM) increased the survival rate for retinal ganglion cells exposed to 50 μM BzATP. Survival was determined 24 hrs after addition of BzATP. Throughout the figures, % RGCs refers to the number of labeled ganglion cells in experimental compared to control conditions, with absolute counts normalized to the mean control for each day. Bars show mean+1 SE. (* diff from BzATP alone, p<0.001, One-way ANOVA with Tukey post-test, n=24 for each). B. Treatment with adenosine (300 μM) also protected cells from the lethal effects of 100 μM NMDA (* diff from NMDA alone, p<0.05, One-way ANOVA Tukey post-test, n=16).

FIG. 9 shows that stimulation of the A₃ receptor inhibits the Ca²⁺ response A. The A3 adenosine receptor agonist CI-IB-MECA prevented the rise in Ca²⁺ triggered by 50 μM BzATP. Cells were exposed to 100 nM CI-IB-MECA for 3 min before and 2 min after application of BzATP to ensure blockage. A small rise in Ca²⁺ can be detected following application of BzATP in the presence of CI-IB-MECA but this is minimal. B. Quantification of the block by CI-IB-MECA. The mean response from 3 separate experiments comparing the peak Ca²⁺ elevation triggered by 50 μM BzATP with the subsequent exposure to BzATP in the presence of 100 nM CI-IB-MECA.

FIG. 10 shows that the A₃ receptor is neuroprotective. A). The loss of ganglion cells following 24 hr incubation with 50 μM BzATP was prevented by co-incubation with 100 nM CI-IB-MECA (CI-IB; n=15; * diff from control, p<0.001; ** diff from BzATP alone, p<0.001). B). Co-incubation of cells with 100 nM IB-MECA (IB) also prevented the death triggered by 50 μM BzATP (n=31-32; * diff from control, p<0.001; ** diff from BzATP alone, p<0.001, Bz+ IB-MECA not diff from control).

FIG. 11 shows Effect of ATP on cell viability wherein incubating cells with ATP (300 μM) for 24 hrs increased the number of retinal ganglion cells as compared to control, while incubating cells with ATPγS (300 μM) reduced cell number. Bars show mean±SE. * diff from control, p<0.05, One-way ANOVA with Tukey post-test, n=32, 14 and 17 for control, ATP and ATPγS respectively.

FIG. 12 shows that expression of the ecto ATPase NTPDase1 can be upregulated in retinal pigmented epithelial cells after exposure to ATPγS. This indicates expression of NTPDase can serve as an index of sustained elevated ATP. It also indicated that upregulation of the enzyme is possible and can be used to increase the conversion of ATP into adenosine. A) demonstrates that the degradation of ATP is increased in RPE cells exposed to ATPγS for 48 hrs. B) Demonstrates that the timeconstant for degradation of ATP falls with increased exposure to ATPγS while C) demonstrates this is significantly different. The increase in protein for NTPDase1 was quantified with antibody Bu61 and demonstrates that increase was also related to exposure to ATPγS, while E) indicates that the exposure led to a rise in mRNA specific for NTPDase1 but not actin.

FIG. 13 shows pressure triggering a release of ATP from the bovine eyecup. A) A significant increase in the ATP concentration is detected in the vitreous humor obtained after the exposure of the retina eyecup to 20 mm Hg of extracellular pressure for 10 minutes (black bar) versus the non-pressured eyecup control samples (white bar). B) The ATP concentration in the retina eyecup after 20 minutes of pressure challenge increased linearly with pressure between 20 and 100 mm Hg (r²=0.947). C) 30 μM NPPB (grey bar) inhibits the ATP release induced by a 20 mm Hg rise in extracellular pressure for 10 minutes (black bar). The ATP levels were normalized to control levels (white bar). D) Increasing pressure by introducing air into the chamber (while bar) had the same effect as injecting N₂ (black bar) indicating a change in partial pressure did not underlie the ATP release. E) Extracellular LDH levels are not significant increased in samples from retina eyecup challenged with 20 (black bar) and 50 mm Hg (grey bar) for 10 minutes versus the control levels in samples collected from non-pressure eyecups (white bar), indicating the increased ATP did not result from damaged cells. The bars and circles represent the mean±SEM. (*=p<0.05, one way ANOVA with Tuckey post-test)

FIG. 14 shows that increase in NTPDase1 is linked to increase in IOP in primate model of chronic glaucoma. Primates had pressure elevated in one eye after receiving laser trabeularotamy. NTPDase1 levels in the retina were compared between lasered and control eyes of the same animal and compared to the change in pressure between the eyes. Of 13 lasered eyes, 12 had increased levels of NTPDase1, as determined using the antibody BU61 on Western blots. The relative increase in NTPDase protein was proportional to elevated pressure.

### DETAILED DESCRIPTION OF THE INVENTION

This invention relates in one embodiment to the role of P2X₇ receptor present on retinal ganglion cells and the associated complex cascade it initiates in suppressing, preventing, inhibiting or reducing the death or apoptosis or disruption of ganglion cells and their ability to act as neuroprotective agents for the treatment of glaucoma.

In one embodiment elevated intraocular pressure (IOP) and cell death are linked. In one embodiment physiologic release of ATP from non-neuronal tissues is triggered by mechanical distention due to swelling or stretching or, in other embodiments release of ATP is a general response to mechano-sensory distension of multiple tissues. In one embodiment, the sustained elevated pressure associated with glaucoma leads to ATP release.

In one embodiment, series of extracellular enzymes serve to dephosphorylate released ATP to produce adenosine in the extracellular space. The adenosine thus formed can activate, in one embodiment the signaling pathways by stimulating the P1 receptors. The molecular sequences and downstream connectivity of the P1 and P2 receptors are distinct, and their stimulation has in another embodiment, diverse consequences for cellular function. The production of discrete responses from ATP and adenosine provide in one embodiment a mechanism for temporal integration of the purinergic signal.

Extensive pharmacologic and physiologic characterization confirms the P2X₇ receptor as mediating the response. While isolated stimulation of this receptor is clearly toxic, the functional implications of receptor activation may be balanced in one embodiment, by the actions of P1 receptors. There are four main families of P1 receptors; namely the A1, A2A, A2B and A3 receptors. Stimulation of both A₁ and A₃ receptors protects cells in another embodiment from insults such as ischemia.

A₃ adenosine receptor contributes to the effect on both Ca²⁺ levels and cell survival. In one embodiment, hydrolysable ATP is protective while non-hydrolysable ATPγS kills ganglion cells at a rate comparable to BzATP. These findings provide a novel structure-function evidence for the effect of A₃ receptor activity on retinal ganglion cells and show that stimulation of P1 receptors counterbalances the detrimental effects of P2 receptor activation on retinal ganglion cells.

According to this aspect of the invention, and in one embodiment, the invention provides a an adenosine A₃ receptor agonist, an adenosine A₁ receptor agonist, an agent capable of blocking the release of excessive ATP in response to elevated intraocular pressure, an ecto-nucleotidase agonist, a Ca²⁺ chelating agent, an NMDA receptor antagonist or their combination, as well as in another embodiment, a pharmaceutically acceptable carrier, excipient, flow agent, processing aid, a diluent or a combination thereof.

In another embodiment, the P2X₇ antagonist used in the compositions and methods of the invention is calmidizolamide in one embodiment, or oxidated Adenosine 5′ triphosphate (OxATP) in another embodiment, or Brilliant Blue G, KN62, KN04 or a combination thereof in other embodiments. In one embodiment, the P2X₇ is a hP2X7-specific monoclonal antibody (MoAb); and combination thereof in other embodiments.

In one embodiment, excitatory amino acids such as glutamate kill neurons. Overstimulation of the NMDA receptor (NMDAR) leads to excessive Ca²⁺influx, activation of apoptotic processes and death of many neuronal types including, in another embodiment, retinal ganglion cells (RGC's). As these steps paralleled those accompanying P2X₇R activation, in one embodiment, NMDAR is involved in the death of retinal ganglion cells following stimulation of the P2X₇R and reducing NMDAR activity using the methods and compositions described herein, is effective in inhibiting or suppressing RGC's death.

Glutamate receptors are characterized in another embodiment, by their sensitivity to specific glutamate analogues and by specific features of the glutamate-elicited currents. In one embodiment, ionotropic glutamate receptors mediate fast synaptic transmission between neurons by forming a single complex between the receptors and the ion channel. NMDA receptors (NMDAR's), bind glutamate and the glutamate analogue N-methyl-D-aspartate (NMDA) with the high conductance channel associated with the NMDA receptors being permeable to Ca²⁺ as well as to Na⁺ and K⁺. NMDA-gated currents have in one embodiment, a slower kinetics than kainate- and AMPA-gated channels.

NMDA receptors are heteromeric ion channels composed of one NR1 subunit (whose presence is mandatory), NR2A-D, and, in some cases, NR3A or B subunits. The receptor is composed in one embodiment, of a tetramer of these subunits. In another embodiment, the subunit composition determines the pharmacology and other parameters of the receptor-ion channel complex. Alternative splicing of some subunits, such as NR1, contributes in one embodiment to the pharmacological properties of the receptor. The subunits are differentially expressed and in one embodiment, the antagonists used in the compositions and methods described herein, are, antagonists-specific for the receptor configuration present on retinal ganglion cells.

Excessive activation of the NMDA receptor in particular leads in another embodiment to production of damaging free radicals and other enzymatic processes contributing to cell death. With the disruption of energy metabolism during acute and chronic neurodegenerative disorders, glutamate is not cleared properly and sufficiently and may even be inappropriately released. Moreover, energetically compromised neurons become depolarized (more positively charged) because of the fact that in the absence of energy they cannot maintain ionic homeostasis; this depolarization relieves the normal Mg²⁺ block of NMDA receptor-coupled channels because the relatively positive charge in the cell repels positively-charged Mg²⁺ from the channel pore.

Adenosine is a naturally occurring nucleoside that exerts its biological effects by interacting with a family of adenosine receptors identified as the adenosine A₁, A₂ₐ, A_{2b}, and A₃ receptors modulate a variety of biological processes. In one embodiment, compounds that are A₁, A₃ adenosine receptor agonists or their combination have utility in the therapeutic and/or prophylactic compositions and methods described herein.

In one embodiment, the compositions described herein, used in the invention further comprise a carrier, or excipient, lubricant, flow aid, processing aid or diluent in other embodiments, wherein the carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal opthalmic and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials. In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with other therapeutical agents. Representative agents that can be used in combination with the compositions of the is invention are agents used to treat diabetes such as insulin and insulin analogs (e.g. LysPro insulin); GLP-1 (7-37) (insulinotropin) and GLP-1 (7-36)—NH.sub.2; biguanides: metformin, phenformin, buformin; .alpha.2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; .alpha.-glucosidase inhibitors: acarbose, miglitol, emiglitate, voglibose, MDL-25,637, camiglibose, MDL-73,945; , .beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan®)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (Symlin™), AC2993, glycogen phosphorylase inhibitor and nateglinide. Any combination of agents can be administered as described hereinabove.

The use of compositions described herein for administration in the methods of treatment described herein, is done in one embodiment via an ophthalmic solution. The solution comprises in one embodiment, aqueous solutions and water-miscible ointments in which the compositions of the invention may be dissolved or suspended in, in finely divided form. The aqueous solutions and suspensions may incorporate pharmaceutically acceptable auxiliary ingredients that are not incompatible with the compositions described herein. A suitable vehicle comprise in another embodiment, a simple physiological saline solution containing 0.9% sodium chloride by weight. Such a solution is isotonic with tear fluid and is therefore non-irritating to the eye. Other solutions or suspensions wherein the formulation including the compositions of the invention and other auxiliary ingredients is hypotonic may be adjusted in one embodiment, to isotonicity by addition of a tonicity adjusting agent, e.g., sodium chloride. In one embodiment, hypotonic and hypertonic solutions or suspensions are also used, and are also acceptable for compliant ocular use. The ophthalmic solutions and suspensions of the invention incorporate in another embodiment other auxiliary agents such as buffers to control the pH within the practical range for storing and applying topical ophthalmic compositions of the inventions, i.e, from about pH 3 to about pH 8.5. In one embodiment, a physiological saline solution is buffered with a suitable buffering agent, e.g., a to phosphate buffer, to maintain approximately physiological pH. Such a solution is buffered in another embodiment, at a pH of 7.2-7.4 to match the natural pH of the tears bathing the anterior segment of the eyeball.

The ophthalmic solution or suspension may incorporate in another embodiment conventional ingredients to improve the comfort of the dosage form, e.g., demulcents, such as polysorbate 80, polyethylene glycol (PEG) 400, dextran 70, gelatin, glycerin, propylene glycol, and the like. The ophthalmic solution or suspension may contain viscosity increasing constituents such as methylcellulose, hydroxypropyl cellulose, hydroxypropyl methylcellulose, poly(vinylpyrrolidone), polyvinyl alcohol, and the like. Several of the viscosity-adjusting agents also exhibit a demulcent function. Many of the viscosity-adjusting agents, when used as constituents of suspensions or emulsions containing the active ingredient, act as suspending agents to retard settling of solid particles or as protective colloids for emulsions to prevent phase separation.

The ophthalmic vehicle, whether hydrophilic in one embodiment, or hydrophobic in another, may also incorporate conventional antimicrobial preservative agents in order to prevent contamination of multiple-dose packages of the ophthalmic medication such as dropping bottles, tubes of ointments or bottles with accompanying eyedroppers. Suitable preservatives include in one embodiment quaternary ammonium compounds, e.g., benzalkonium chloride, cetylpyridinium chloride and the like; ethyl paraben, propyl paraben; alcohols, such as benzyl alcohol; organomercurial compounds, such as thimerosal; polybiguanide compounds such as chlorhexidine digluconate, polyaminopropyl biguanide, and the like. A compound that promotes the permeation of the compositions of the invention into the ocular tissues, such as dimethyl sulfoxide, a quaternary ammonium compound, e.g., benzalkonium chloride, or an opthalmologically acceptable surfactant, e.g., disodium lauryl sulfosuccinate, or the like may also be incorporated into the ophthalmic vehicle. When the composition of the invention is administered in the form of a suspension in an aqueous medium the suspension may also contain a suspending agent, e.g., methyl cellulose, propylcellulose, carboxmethyl-cellulose, poly(vinylpyrrolidone), poly(vinyl alcohol), and the like.

In one embodiment, the compositions described herein are used to bind and occupy the P2X₇ receptor. P2X₇ receptor refers in one embodiment to a ligand-gated ion channel that is activated by extracellular ATP. Its activation results in one embodiment, in the opening of a cationic channel with significant permeability to calcium, loss of cross-membrane potential and intracellular depolarization. In another embodiment, P2X₇ receptor is bifunctional. Brief stimulation by low concentrations of agonist in one embodiment, leads to the receptor acting as a nonselective cation channel. In another embodiment, repeated, sustained or prolonged application of higher agonist concentrations, such as in one embodiment, in solutions containing low concentrations of extracellular divalent cations, creates a much larger aqueous pore. In one embodiment ganglion cells exhibit the same responses to ATP. In one embodiment, P2X₇ to receptors mediate ATP-induced cell death and the compositions described herein, which are used in the methods described herein comprising at wo of a P2X₇ antagonist, a Ca²⁺ chelating agent, an NMDA receptor antagonist, adenosine A3 receptor agonist or a combination thereof, reduce, suppress, inhibit or ameliorate cell death.

In one embodiment, the term “antagonist” in the context of describing compounds according to the invention refers to a compound that directly or in another embodiment, indirectly inhibits, or in another embodiment suppresses receptor activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the receptor, even when there are spare (unbound) receptors present. In another embodiment, the term “partial antagonist” refers to a compound does not evoke the maximal inhibitory response from the androgen receptor, even when present at concentrations sufficient to saturate the androgen receptors present.

In another embodiment, the antagonists used in the methods and compositions of the invention, are uncompetitive antagonists. The term “uncompetitive antagonists” refer in one embodiment to an inhibitor whose action is contingent upon prior activation of the receptor by the agonist. Hence, in one embodiment, the same amount of antagonist blocks higher concentrations of agonist better than lower concentrations of agonist. This uncompetitive mechanism of action, coupled with a longer dwell time than Ca²⁺in the channel (and consequently a slower “off-rate” from the channel) but a substantially shorter dwell time receptor-operated channels only when they are excessively open while relatively sparing normal neurotransmission.

In another embodiment, the term “agonist” in the context of describing compounds according to the invention, refers to a compound that when bound to the receptor, enhances or increases the receptor receptor activity, function, ligand mediated transcriptional activation, or in another embodiment, signal transduction through the receptor. As such, agonists include partial agonists and full agonists. In another embodiment, the term “full agonist” refers to a compound that evokes the maximal response from the receptor, even when there are spare (unoccupied) receptors present. In one embodiment, the term “partial agonist” refers to a compound that is unable to evoke the maximal stimulatory response from the receptor, even at concentrations sufficient to saturate the P2X₇ receptors present.

In one embodiment, the invention provides a composition comprising a P2X₇ antagonist wherein the antagonist is calmidizolamide in one embodiment, or oxidated Adenosine 5′ triphosphate (OxATP) in another embodiment, or Brilliant Blue G, KN62, KN04 or a combination thereof in other embodiments. In one embodiment, the P2X₇ is a hP2X7-specific monoclonal antibody (MoAb). In one embodiment, the agonist used in the methods and compositions described herein, is an agonist of adenosine A1, A3 receptor or their combination. In one embodiment, the agonist may be the same for both receptors, or different.

In one embodiment, the term “antibody” include complete antibodies (e.g., bivalent IgG, pentavalent IgM) or fragments of antibodies in other embodiments, which contain an antigen binding site. Such fragment include in one embodiment Fab, F(ab′)₂, Fv and single chain Fv (scFv) fragments. In one embodiment, such fragments may or may not include antibody constant domains. In another embodiment, F(ab)'s lack constant domains which are required for complement fixation. scFvs are composed of an antibody variable light chain (V_{L}) linked to a variable heavy chain (V_{H}) by a flexible linker. scFvs are able to bind antigen and can be rapidly produced in bacteria. The invention includes antibodies and antibody fragments which are produced in bacteria and in mammalian cell culture. An antibody obtained from a bacteriophage library can be a complete antibody or an antibody fragment. In one embodiment, the domains present in such a library are heavy chain variable domains (V_{H}) and light chain variable domains (V_{L}) which together comprise Fv or scFv, with the addition, in another embodiment, of a heavy chain constant domain (C_{H1}) and a light chain constant domain (C_{L}). The four domains (i.e., V_{H}-C_{H1} and V_{L}-C_{L}) comprise an Fab. Complete antibodies are obtained in one embodiment, from such a library by replacing missing constant domains once a desired V_{H}-V_{L} combination has been identified.

The antibodies described herein can be monoclonal antibodies (Mab) in one embodiment, or polyclonal antibodies in another embodiment. Antibodies of the invention which are useful for the compositions, methods and contraceptives described herein can be from any source, and in addition may be chimeric. In one embodiment, sources of antibodies can be from a mouse, or a rat, or a human in other embodiments. Antibodies of the invention which are useful for the compositions, methods and contraceptives of the invention have reduced antigenicity in humans, and in another embodiment, are not antigenic in humans. Chimeric antibodies as described herein contain in one embodiment, human amino acid sequences and include humanized antibodies which are non-human antibodies substituted with sequences of human origin to reduce or eliminate immunogenicity, but which retain the binding characteristics of the non-human antibody. In one embodiment, the antibody used to inhibit activity of P2X₇, is a hP2X₇-specific monoclonal antibody (MoAb).

In certain embodiments, the antibodies employed in the compositions described herein and used in the methods described herein, will be “humanized”, part-human or human antibodies. In one embodiment, “Humanized” antibodies are generally chimeric monoclonal antibodies from mouse, rat, or other non-human species, bearing human constant and/or variable region domains (“part-human chimeric antibodies”). Various humanized monoclonal antibodies for use in the present invention will be chimeric antibodies wherein at least a first antigen binding region, or complementarity determining region (CDR), of a mouse, rat or other non-human monoclonal antibody is operatively attached to, or “grafted” onto, a human antibody constant region or “framework”.

“Humanized” monoclonal antibodies for use herein may also be monoclonal antibodies from non-to human species wherein one or more selected amino acids have been exchanged for amino acids more commonly observed in human antibodies. This can be readily achieved through the use of routine recombinant technology, particularly site-specific mutagenesis.

Inward currents evoked by BzATP were inhibited in one embodiment, by contacting the cells with hP2X₇-specific monoclonal antibody (MoAb). In another embodiment, this inhibition is concentration-dependent, and currents are reduced to approximately half. Blockade of the human P2X₇ receptor by the MoAb reversible in another embodiment, such that after 30 minutes of washing, agonist-evoked inward currents are still inhibited. In another embodiment, incubation of ganglion cells with the MoAb causes a concentration-dependent inhibition of IL-1β, release, such that significant inhibition of the BzATP-induced release could be obtained with the MoAb.

In one embodiment, the antibody, a fragment thereof, or their combination, exhibit substantially complimentarily to their target sequence, which may be a protein, such as P2X₇ receptor protein. In another embodiment, “complementary” indicates that the oligopeptide has a base sequence containing at least 15 contiguous base region that is at least 70% complementary, or in another embodiment at least 80% complementary, or in another embodiment at least 90% complementary, or in another embodiment 100% complementary to an-at least 15 contiguous base region present on a target protein sequence (excluding RNA and DNA equivalents). The degree of complementarity is determined by comparing the order of nucleobases making up the two sequences and does not take into consideration other structural differences which may exist between the two sequences, provided the structural differences do not prevent hydrogen bonding with complementary bases. The degree of complementarity between two sequences can also be expressed in terms of the number of base mismatches present in each set of at least 15 contiguous bases being compared, which may range from 0-3 base mismatches, so long as their functionality for the purpose used is not compromised.

An antibody with an ability to inhibit human P2X₇ receptor will generally exhibit a consistently observed inhibition of human P2X₇ receptor of about 25%, 30%, 35%, 40% 45% or 50% or so. Inhibition in such ranges will indicate an antibody with properties sufficient to inhibit glaucoma, or chronic glaucoma in vivo. Antibodies with more significant inhibitory activity are not excluded from the scope of the invention.

As will be understood by those skilled in the art, the immunologically binding reagents encompassed by the term “antibody” extend in certain embodiments, to all antibodies from all species including dimeric, trimeric and multimeric antibodies; bispecific antibodies; chimeric antibodies; human and humanized antibodies; recombinant and engineered antibodies, and fragments thereof. The term “antibody” is refers in another embodiment to any antibody-like molecule that has an antigen binding region, and this term includes antibody fragments such as Fab′, Fab, F(ab′).sub.2, single domain antibodies (DABs), Fv, scFv (single chain Fv), linear antibodies, diabodies, and the like. The techniques for preparing and using various antibody-based constructs and fragments are well known in the art (see Kabat et al., 1991, specifically incorporated herein by reference).

The term “antibody fragment” also includes any synthetic or genetically engineered protein that acts like an antibody by binding to a specific antigen to form a complex. In one embodiment, antibody fragments include isolated fragments, “Fv” fragments, consisting of the variable regions of the heavy and light chains, recombinant single chain polypeptide molecules in which light and heavy chain variable regions are connected by a peptide linker (“sFv proteins”), and minimal recognition units consisting of the amino acid residues that mimic the hypervariable region. In one embodiment, the antibody capable of inhibiting human P2X₇ receptor is a variable regions of the heavy and light chains, or recombinant single chain polypeptide molecules in which light and heavy chain variable regions are connected by a peptide linker (“sFv proteins”), and minimal recognition units consisting of the amino acid residues that mimic the hypervariable region in other embodiments.

In another embodiment, the invention provides a composition comprising an NMDA receptor antagonist wherein the antagonist is memantine.

In one embodiment, stimulation of the P2X7 receptor in retinal ganglion cells leads to release of glutamate which elevates intracellular Ca²⁺ levels and kills the neurons. In another embodiment, the ability of NMDAR antagonists which act at distinct sites on the NMDA protein to block BzATP response indicates that the block is specific for the NMDAR. In one embodiment, the relative effectiveness of MK-801, APV, memantine or their combination at blocking the response is similar in ganglion cells from both mixed retinal cells and isolated ganglion cell preparations, and is analogous to the strength of their block at the NMDA receptors. In another embodiment, the ability of NMDAR antagonists to reduce cell death triggered by BzATP indicates a role for the NMDAR downstream from the P2X7R. The ability of BzATP to trigger glutamate release into the bath provides direct evidence that the purinergic and glutaminergic systems are linked. The time course of the glutamate efflux correlates in one embodiment closely with the Ca²⁺ elevations in response to BzATP, with the reversible and repeatable nature of both responses implying the two are related.

In one embodiment, the release of glutamate following BzATP stimulation distinguishes the downstream activation of the NMDA receptor by the P2X7R from the more commonly known actions of the AMPA receptor. The NMDA receptor is closed in another embodiment, at the resting membrane potential even in the presence of agonist, but the influx of cations following activation of AMPA/kianate receptors by glutamate depolarizes the neurons and relieves the voltage-dependent Mg block. For cell viability experiments, cells are maintained in one embodiment, in neural culture media containing 0.8 mM Mg²⁺ and the influx of cations through the P2X₇ channel relieves the Mg²⁺ block.

In one embodiment, the partial block of the Ca²⁺ response by MK-801 indicates both the P2X7 and NMDA receptors contribute to the Ca²⁺ response. Complete restoration of cell numbers by MK-801 in another embodiment, indicates that the opening of the NMDA receptor is necessary for cell death. The specific ability of NMDA receptor activation to kill cells is of particular interest, with linkage to specific lethal targets through cytoplasmic PDZ domains proposed to distinguish the NMDAR response. In another embodiment L-type Ca²⁺ channel blocked nifedipine somewhat reduced cell death due to BzATP. As both the NMDAR and P2X7 R cause in one embodiment, a secondary activation of voltage-dependent Ca²⁺ channels. In another embodiment, functional characterization including the relative efficacy of BzATP vs ATP and the ability of brilliant Blue G to and KN04 inhibit the response at low levels are consistent only with the presence of the P2X7 receptor. The enhancement of the Ca²⁺ response to BzATP following Mg2+ removal in one embodiment, is consistent with P2X7 receptor, reflecting the block of the NMDA channel by Mg²⁺.

In one embodiment, co-localization of both P2X7 and NMDA receptors on adult ganglion cells and ability of NMDA antagonists to prevent the death of adult ganglion cells by BzATP indictaes interaction between co localization of the P2X7 receptor and NMDA receptors persists into maturity.

In one embodiment, the Adenosine A3 receptor agonist used in the compositions and methods of the invention, is adenosine (ADO), or 2-chloro-N6-(3-iodobenzyl)-adenosine-5-N-methyluronamide (CI-IB-MECA), or a combination thereof in other embodiments. The A3 agonists may be used alone or in conjunction with A1 receptor agonists.

The ability of adenosine and CI-IB-MECA to both limit the increase in Ca²⁺ and stop cell death indicates that the increase in Ca²⁺is a necessary step in ganglion cell death following P2X₇ receptor activation. Excess elevation in Ca²⁺ can lead to death of neurons with mitochondieal depolarization and activation of apoptotic transcription factors, consistent with a downstream activation of endonucleases and proteases typically observed in Ca²⁺ mediated apotosis. The central role of Ca²⁺ elevation in ganglion cell death triggered by NMDA is supported by the observation that inhibition of L-type Ca²⁺ channels with dihydropyridine prevented cell loss.

In one embodiment, stimulation of the A₃ receptor counteracts the destructive actions of P2X₇ receptor stimulation. In another embodiment, the elevated levels of extracellular ATP contribute to ganglion cell death in glaucoma and A3 agonists are protective. A variety of enzymes are responsible for the conversion of extracellular ATP into adenosine; enhancement of such enzyme activity would simultaneously limit actions of ATP while increasing available adenosine and represent a viable neuroprotective approach in glaucoma and other optic neuropathies. In one embodiment, the compositions and methods of the invention are used to treat glaucoma and optic neuropathies characterized in another embodiment, by cupping of the optic nerve head, thinning of the retinal nerve fiber layer due to loss of retinal ganglion cells, and specific pathognomonic changes in visual fields, such as in one embodiment ocular hypertension.

In one embodiment, the compositions described hereinabove are used in the methods described herein. In another embodiment, the invention provides a method for inhibiting or suppressing the reduction in number of retinal ganglion cells in a subject, comprising administering to said subject an effective amount of a P2X₇ antagonist, thereby preventing the stimulation of the receptor leading to death of ganglion cells and a reduction in their numbers.

In one embodiment, cross membrane potential refers to the electrophysiological properties of the RGC's membrane, such as current flow through an ion channel, or electric potential across an ion channel, or capacitance or impedance of an ion channel containing membrane in other embodiments. In another embodiment, transmembrane ion gradients result in imposed cross-membrane potential difference, which, when sustained increase in ATP activates P2X₇ receptor, results in loss of the abovementioned transmembrane ion gradient, due to the opening of a non-selective ion channel as described herein. The resultant depolarization of plasma membranes leads to Ca²⁺influx through voltage-dependent Ca²⁺ channels. A steep rise in intracellular Ca²⁺ ([Ca²⁺]c) is buffered to some degree by mitochondrial Ca²⁺ uptake. However, once a continuous increase in [Ca²⁺]c exceeds the buffering capacity, these organelles become dysfunctional via opening of a nonspecific pore in the mitochondrial membrane, the permeability transition (PT) pore. Mitochondrial Ca²⁺ overload seems to be a consequence of the rise in the cytosolic Ca²⁺ concentration promoted by Ca²⁺ entry through plasma membrane receptor-operated and voltage-dependent Ca²⁺ channels. The mitochondrial dysfunction contributes in one embodiment to apoptotic cell death.

Elevation of Ca²⁺i is in one embodiment, an essential early step in the cell body-mediated death, and this Ca²⁺i increase induces in another embodiment, apoptotic loss by activation of endonucleases and proteases. Inhibition of Ca²⁺ channels prevents in one embodiment, ganglion cell loss proving the role of Ca²⁺i elevation in ganglion cell death. Therefore, removing the receptor agonis BzATP, will in one embodiment reduce the concentration of Ca²⁺i and suppress, or inhibit ganglion cell loss. In one embodiment, the agonist removed is Ca²⁺ and removal is done by administrating a chelating agent.

In one embodiment, ATP, by acting at plasma membrane P2 receptors of which P2X₇ receptor is a member, triggers different cell responses, such as secretion, chemotaxis, proliferation, transcription factor activation, or cytotoxicity. In another embodiment, ATP is a powerful apoptotic agent via activation of the purinergic P2X₇ receptor, capable of generating a nonselective pore or activating the excitotoxic processes through the NMDA receptor upon sustained stimulation. In one embodiment P2X7 expression causes excess ca2+ influx in response to ATP. Therefore, removal of ATP will suppress cell death caused by P2X₇ receptor activation. In one embodiment this removal is performed by adding soluble ecto-nucleotidases or by increasing expression of endogenous ecto-nucleotidases such as NTPDase1.

In another embodiment, any of the methods of suppressing, or inhibiting the loss or death or loss of function of ganglion cells in the retina, as described hereinabove, are useful as neuroprotective method for protecting the optic nerve and are therefore useful in treating Glaucoma in a subject.

In one embodiment, provided herein is a method of treating a pathological condition in a subject resulting from a reduction in number of retinal ganglion cells, comprising administering to said subject the composition of claim 1, thereby preventing the opening of a the receptor leading to death of ganglion cells, a reduction in their number thereby resulting in loss of function of said retinal ganglion cells. In another embodiment, the pathological condition resulting from decrease in number of RGCs is glaucoma, or chronic glaucoma.

According to this aspect of the invention and in another embodiment, provided herein is a method for inhibiting or in another embodiment, suppressing or in another embodiment neuroprotecting the reduction in retinal ganglion cells in a subject, where the subject exhibits increase in intraocular pressure. In one embodiment, the increase in intraocular pressure (IOP) is sustained over period of time which induces increased levels of NTPDase1.

Intraocular pressure, refers in one embodiment to the force required to flatten a given area of the cornea, which is proportional to the pressure inside the eye. The most common methods of measurement include Goldmann applanation, a hand-held device known as a Tonopen, and pneumo-tonometry. Applanation tonometry (Goldmann applanation and Tonopen) is performed after anesthetizing the ocular surface with a topical anesthetic medication. Normal eye pressures ranges in one embodiment from about 10 to 21 mm H_{g} and has a diurnal variation.

Glaucoma affects 2 million Americans, and half are unaware of the disease. Approximately 5 to 10 million Americans have elevated eye pressure, placing them at risk for the development of glaucoma. Eighty thousand Americans are already blind from the disease. African-Americans have a five-fold greater risk of developing glaucoma and, in this population, it is the single most common cause of irreversible blindness. Glaucoma, is a myriad of diseases with a final common result, injury to the optic nerve. Therefore, it is the purpose of this invention in one embodiment, to treat Glaucoma through the neuroprotection of the optic nerve.

According to this aspect of the invention, and in one embodiment, the invention provides method of treating glaucoma in a subject, comprising administering to said subject an effective amount of a A3 agonist or P2X₇ antagonist. In another embodiment, any of the compositions described herein are useful in treating chronic glaucoma in a subject.

In one embodiment, the invention provides a method for enhancing the conversion of ATP into adenosine outside of a retinal cell, comprising: increasing activity for ecto-nucleotides; and removing ATP thereby producing adenosine.

In another embodiment, increasing the activity of ecto-nucleotides according to the methods of the invention, comprises contacting the cell with a purinergic agonist, thereby upregulating expression of the gene encoding for ecto-nucleoside triphosphate diphosphohydrolase (NTPDase)1. In one embodiment, the purinergic agonist is ATPγS. In one embodiment, an “agonist” refers to a ligand, that activates an intracellular response when it binds to a receptor at concentrations equal or lower to ADP concentrations which induce an intracellular response. An agonist according to the invention may increase the intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc . . . ), as compared to the intracellular response in the absence of agonist. An agonist, according to the invention may decrease internalization of a cell surface receptor such that the cell surface expression of a receptor is increased by at least 2-fold, preferably 5-fold, more preferably 10-fold and most preferably, 100-fold or more (i.e., 150-fold, 200-fold, 250-fold, 500-fold, 1000-fold, 10,000-fold etc . . . ), as compared to the number of cell surface receptors present on the surface of a cell in the absence of an agonist. In another embodiment of the invention, an agonist stabilizes a cell surface receptor and increases the cell surface expression of a receptor by at least 2-fold, preferably 5-fold, more preferably 10-fold and most preferably, 100-fold or more (i.e., 200-fold, 250-fold, 500-fold, 1000-fold, 10,000-fold etc . . . ), as compared to the number of cell surface receptors present on the surface of a cell in the absence of agonist.

In one embodiment, Adenosine 5′-O-[3-thiotriphosphate] (ATPγS) is a nonhydrolyzable ATP analog that weakly activates the P2X7 receptor.

In one embodiment, the invention provides a method of reducing the release of cytotoxic ATP from a retinal cell in response to elevated intraocular pressure, comprising contacting said cell with a Cl⁻ and/or hemichannel blocker. In another embodiment, the Cl⁻ channel blocker is NPPB (5-nitro-2-(3-phenylpropyl-amino)benzoic acid), or SITS (4-acetamido-4′-isothiocyanostilbene-2,2′-disulphonic acid), NFA (niflumic acid), DIDS (4,4′-diisothiocyanatostilbene-2,2′-disulfonic acid), A9C (anthracene-9-carboxylic acid), N-phenylanthranilic acid, DPC (diphenylamine-2-carboxylic acid), IAA-94 (R(+)methylindazone, indanyloxyacetic acid 94), 2-aminomethyl phenols, MK-447 (2-aminomethyl-4-(1,1-dimethyl ethyl)-6-iodophenol hydrochloride (2) disulfonic stilbenes, or a combination thereof in other embodiments used in the methods of the invention. In one embodiment, siRNA for voltage dependent anion channel or volume selective osmolyte channels delivered to retinal glial cells, are used as part of the methods and compositions of the invention, as identified as route for ATP release and may be used to prevent in one embodiment, or reduce in another embodiment, the secretion of ATP.

In one embodiment, gap junctions connect the cytoplasm of adjacent cells, allowing ionic and metabolic exchange between them and mediating metabolic cooperation thereby optimizing the functioning of many tissues, including in another embodiment, retinal ganglion cells. Gap junctions are formed in another embodiment, of connexins, a family of homologous protein subunits, and their channels are connexin dodecamers formed of hexameric hemichannels, one from each of the coupled cells. In one embodiment, open hemichannels in nonjunctional membrane have permeability properties similar to those of the intercellular channels. In another embodiment, under physiological conditions, unapposed hemichannels are closed using the blockers described herein, thereby preventing metabolic stress and death caused by the collapse of ionic gradients, loss of small metabolites, and influx of Ca²⁺ or their combination. In another embodiment, the hemichannel blockers are mefloquine acid, meclofenamic acid, retinoic acid, 18-α-glycyrrhetinic acid, flufenamic acid, niflumic acid, carbenoxolone and connexin mimetic peptides or their combination in other embodiments.

In another embodiment, the increase in IOP results in release of cytotoxic ATP from a retinal cell in response to the elevated intraocular pressure, and contacting the retinal cells with a channel blockers, will reduce the release of cytotoxic ATP. In one embodiment, provided herein is a method of reducing the release of cytotoxic ATP from a retinal cell in response to elevated intraocular pressure, comprising contacting said cell with an inhibitor of ATP release, thereby decreasing the release of excess ATP into the retina in response to elevated pressure. In one embodiment, the inhibitor of ATP release, used in the methods and compositions described herein, is a Cl⁻ channel, hemichannel blocker or a combination thereof.

In one embodiment, “contacting” a cell with a substance refers to (a) providing the substance to the environment of the cell (e.g., solution, in vitro culture medium, anatomic fluid or tissue) or (b) applying or providing the substance directly to the surface of the cell, in either case so that the substance comes in contact with the surface of the cell in a manner allowing for biological interactions between the cell and the substance.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Methods

### Retinal Cell Culture and Labeling of RGCs

Pups PD2-6 from untimed pregnant Long-Evan rats (Jackson Laboratory Inc., Bar Harbor, Me.) were back-labeled by the injection of FluoroGold derivative aminostilbamidine (Molecular Probes, Eugene, Oreg.) based upon standard protocols. Pups were anesthetized with an i.p. injection of 50/5 mg/kg ketamine/xylazine, an incision exposed the skull and a 1 mm hole was drilled through the skull, exposing the cortex overlying each superior colliculus. Using a Hamilton syringe affixed to a micromanipulator, a needle was inserted 0.8 mm lateral from the midline and 0.8 mm anterior to Bregma's line and a total of 2.5 μl dye was delivered to each side at a depth of 2 mm and 1 mm. The needle was retracted after a delay of 2 min to allow dye absorption and the wound was closed with 2-3 sutures. Preliminary examination of labeled retinal whole mounts confirmed an even distribution of dye, showing all cells were stained 2 days after injection, with no further increase in the number of labeled cells in subsequent days. Consequently, retinas containing labeled ganglion cells were dissociated 2-6 days after injection. Animals were sacrificed by i.p. injection of 50/5 mg/kg Ketamine/Xylazine followed by an overdose, in accordance with University of Pennsylvania IACUC approved protocols and the ARVO Statement on the Use of Animals in Ophthalmic and Vision Research.

### Retinal Culture

The retina was dissected from each globe, washed in sterile Hanks' balanced salt solution (HBSS, Gibco, Inc Invitrogen Corp., Carlsbad, Calif.), then incubated in HBSS containing activated papain (4.5 U/ml; Worthington Biochemical Corp., Lakewood, N.J.) for 12 minutes at 37° C. Retinas were washed twice and triturated 50 times with a 1-ml glass pipette to dissociate cells. Cells were plated onto twelve 12-mm coverslips previously coated with poly-L-lysine. The basic growth medium contained Neurobasal medium with 2 mM glutamine, 100 μg/ml gentamicin, 0.025 ml/ml B27 supplement (all Invitrogen Inc., Carlsbad, Calif.), 0.7% methylcellulose (Stemcell Technologies Inc., Vancouver, BC, Canada) and 2.5% rat serum (Cocalico Biologicals Inc., Reamstown, Pa.). Retinal cells were incubated at 37° C. with 5% CO₂.

### Cell Viability Studies

Drugs were added to the culture medium at the time when cells were plated onto coverslips. After incubation for the indicated time, coverslips were mounted on a Nikon Eclipse E600 microscope equipped for epifluorescence and the fluorescent cells (360±40 nm excitation, >515 nm emission) present in 80 central fields were counted with a 40× objective. All counts were performed in a masked fashion. In experiments involving antagonist pretreatment, drugs were added to the medium at the time of plating. After preincubation at 37° C., stock concentrations of BzATP were added directly to the cells to give the final concentration shown.

### Intracellular Ca²⁺ Measurements

Unlabeled RGCs grown on coverslips for 24 hrs were loaded with 10 μM fura-2 and 2% pluoronic (Molecular Probes, Eugene, Oreg.) for 60-90 min at room temperature, rinsed and maintained in fura-2-free solution for 30 min before data acquisition began. The coverslips were mounted on a Nikon Diaphot inverted microscope and visualized with a 40× objective. Preliminary experiments using cells labeled with aminostilbamidine dye demonstrated that all bright, granulated cells with axonal processes were fluorescent, allowing individual unlabeled cells to be identified upon morphologic criteria. To obtain Ca²⁺ measurements, the field was alternatively excited at 340 nm and 380 nm with a scanning monocrometer and the fluorescence emitted >520 nm from a region of interest surrounding individual retinal ganglion cells was imaged with a CCD camera and analyzed (all Photon Technologies International, Inc., Lawrenceville, N.J.). Cells were perfused with a control solution at the start of Ca²⁺imaging experiments containing (in mM) 105 NaCl, 4.5 KCl, 2.8 NaHepes, 7.2 Hepes acid, 1.3 CaCl2, 0.5 MgCl2, 5 glucose, 75 mannitol, pH 7.4. Drugs were dissolved into the control solution. Calibration was performed separately on each cell after the experiment by perfusing cells in the presence of 5 μM ionomycin and control solution (with 1.3 mM Ca2+) followed by ionomycin in the base solution without Ca²⁺ and with the addition of 5 mM EGTA (pH 7.4). The 340/380 ratio was converted to Ca²⁺ concentration as previously described [1]. All experiments were performed at room temperature.

### Ganglion Cell Panning

Purification of ganglion cells using the panning procedure is based upon published methods (Hartwick et al., 2004). Neonatal rat retinas (PD 7-12) were dissected and incubated at 37° C. for 30 min in HBSS containing 15 U/mL papain, 0.2 mg/mL DLcysteine and 0.004% DNAse I (Worthington/Cooper, Lakewood, N.J.). The tissue was triturated in HBSS with 1.5 mg/ml ovomucoid (Worthington/Cooper, Lakewood, N.J.), 1.5 mg/ml BSA and 0.004% DNase I, centrifuged at 200 g for 11 minutes at room temperature, and cells were rewashed with 10 mg/ml ovomucoid-BSA solution. After centrifugation, cells were resuspended with PBS containing 0.2 mg/ml BSA and 5 μg/ml insulin and filtered through a Nitex mesh (Small Parts Inc, Miami Lakes, Fla.). Cells were incubated with rabbit antimacrophage antibody (1:75, Accurate Chemical, Westbury, N.Y.), then incubated in a 100 mm dish coated with goat anti-rabbit IgG antibody (1:400, Jackson ImmunoResearch Laboratories Inc, West Grove, Pa.). Non-adherent cells were removed to a second petri-dish coated with goat anti-mouse IgM (1; 300, Jackson ImmunoResearch Laboratories Inc, West Grove, Pa.) and anti-Thy 1.1 antibody (from hybridoma Ti D7e2; American Type Culture Collection, Rockville, Md.). After 30 min, non-adherent cells were washed off, and incubated with 0.125% trypsin for 10 min at 37° C. Fetal bovine serum (30%) in neural basal medium was used to stop the digestion and the cells were centrifuged and plated as above on coverslips coated with poly-L-lysine and laminin.

### Glutamate Measurements.

The measurement of glutamate was analogous to that used previously by the Haydon laboratory. The assay is based upon the principle that, in the presence of glutamate, L-glutamic dehydrogenase (GDH) reduces B-nicotinamide adenine dinucleotide (NAD+) to NADH. As NADH fluoresces when excited at 360 nm, this emission provides an index of extracellular glutamate. Coverslips containing ganglion cells purified with the immunopanning technique above, were perfused with isotonic saline solution—supplemented with 56 units/ml L-glutamatic dehydrogenase (GDH) and 1 mM beta-NAD+. After obtaining stable background levels for 40 sec, 10 μM BzATP was added to the perfusate for 20 sec before returning to the supplemented control solution. When multiple measurements were made, cells were washed for 5 min in GDH/NAD-free saline solution to conserve enzyme levels. stimulus to cause glutamate release, whereupon GDH reduces NAD+ to NADH. NADH fluorescence is excited using a xenon arc lamp (100 watts) with a D360/10x exciter filter (Chroma Technology Corp., Brattleboro, Vt.), 510DRLP dichroic mirror (Omega Optical, Brattleboro, Vt.), and 515EFLP emission filter (Omega Optical). Images collected before, during and after the BzATP stimulus indicate the level of fluorescence. The percent increase in fluorescence, figured by dividing the change in fluorescence (ΔF) by the fluorescence level before stimulation (F₀), represents the level of glutamate release. Values were corrected for the decrease shift due to a small perfusion artifact. The correction was minimal, raising the % increase from 15.3 to 17.1%.

### Immunohistochemistry

Data analysis and materials

Data are presented as mean±standard error of the mean. Significance was evaluated using a Student's unpaired t test, with a p value <0.05 signifying significance. For cell viability studies, the number of experiments, n, represents the number of coverslips from which 80 fields were measured and averaged. All values were normalized to the mean control level for that day's matched set of experiments to control for variation in plating efficiency.

In Ca²⁺ experiments, n refers to the number of responses tested. The BzATP concentration-response curve was fit with a standard exponential function y=y0+ae(−bx) using Sigmaplot software (SPSS Inc, Chicago, Ill.). The % block of Ca2+ elevations is defined as 100*(a−b)/a, where a is the response under control conditions and b is the response under experimental conditions. All materials are from Sigma Chemical Corp, (St. Louis, Mo.) unless otherwise indicated.

### Example 1

### Adenosine Prevents Death of Retinal Ganglion Cells Following P2X7 Receptor Activation by Acting at A3 Receptors

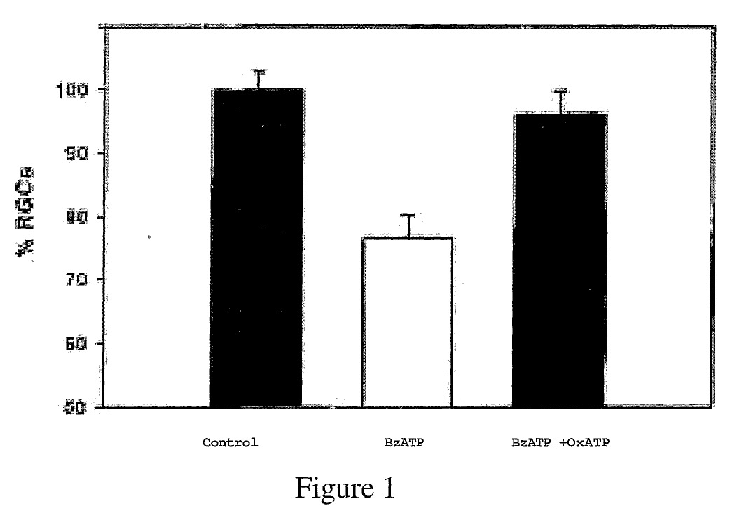

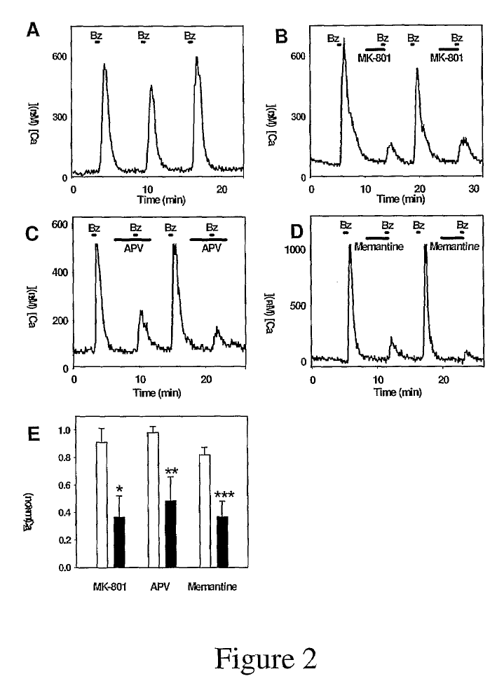

As the excessive influx of Ca2+ accompanying stimulation of the P2X7R in retinal ganglion cells was similar to that observed after N-methyl-D-aspartate (NMDA) receptor activation, it was hypothesized that activation of N-methyl-D-aspartate (NMDA) receptors could occur downstream from P2X7R stimulation. Initial experiments examined whether NMDA antagonists could modify the effect of P2X7R agonist BzATP on neuronal Ca2+ levels. Ganglion cells present in preparations of mixed is retinal cells were examined first. Brief 15 sec applications of BzATP triggered large, reversible and repeatable elevations in the Ca2+ levels of retinal ganglion cells (FIG. 2A). However, the ability of BzATP to increase Ca2+ was attenuated by NMDA antagonists. The presence of the NMDA channel blocker MK-801 in the bath during alternative applications of BzATP decreased the response. (FIG. 2B). When the peak levels were compared to the mean response immediately before and after, 10 μM MK-801 decreased the Ca2+ elevation by 55.1±6.5%. D-amino-phosphonovalerate (APV) also interfered with the ability of BzATP to elevate Ca2+ (FIG. 2C); the mean reduction was of 39.7±12.6%. The effect of memantine (30 μM) was variable, with a 65.5±6.7% reduction in 4/7 experiments (FIG. 2D). The effects of NMDA antagonists on gangion cells from mixed retinal populations are summarized in FIG. 1E.

As shown in FIG. 2, in ganglion cells from mixed retinal cultures, NMDA receptor antagonists reduce Ca²⁺ elevation triggered by P2X₇ receptor activation. (2A) Application of 50 μM BzATP (Bz) for 15 sec led to a large increase in Ca²⁺ levels that returned to normal after removal of BzATP. Duration of drug application is indicted by lines over the trace. Reapplication after 6 min wash led to an elevation similar to the first, with multiple responses evident. (2B) Application of 10 μM MK-801 reduced the Ca²⁺ elevation triggered by 50 μM BzATP. MK-801 was added to the bath 3 min before alternate applications of BzATP and either removed with BzATP as in this example or maintained for an additional 3 min. (2C) The presence of 100 μM 2-APV led to a similar reduction in the response to BzATP. (D) A substantial block of the BzATP response was also found with 100 μM 2-APV (2C) and 30 μM memantine (2D). E. Summary of the effects of NMDA receptor blockers on response to BzATP in ganglion cells from mixed retinal neurons. Bars represent the mean±SEM; Bz is the mean rise to applications 1 and 3 (BzATP alone), while the value for each drug is the mean increase to applications 2 and 4. All values were normalized to the increase detected in the first application of BzATP alone. 10 μM MK-801, N═S; * p=0.019; 300 μM APV, N=5, p=0.033, 30 μM memantine, N=13, *** p=0.002; Students t-test.

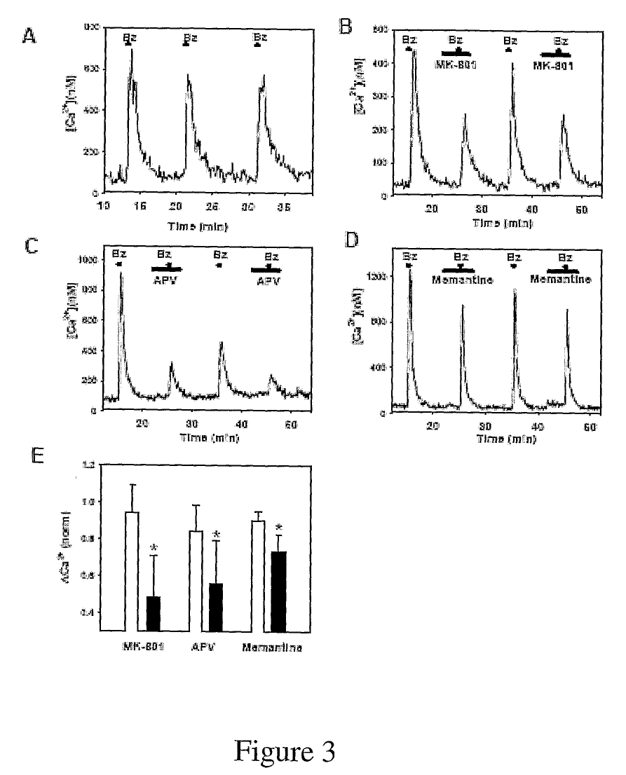

The ability of three different NMDA antagonists with distinct sites of action to block the Ca2+ rise by BzATP made it highly likely that stimulation of the NMDA receptor enhanced the increase in Ca²⁺. However, the above experiments were performed using ganglion cells in a population of mixed retinal cells. As such, it was possible that the interaction between the NMDA and P2X7 receptors required other retinal cell types. To rule out a necessary contribution from other retinal cells, the experiments were repeated with ganglion cells isolated using the immunopanning procedure. These preparations were highly purified, containing >98% ganglion cells. Brief applications of BzATP led to repeatable and reversible elevations in cell Ca2+ similar to those observed previously in cells from mixed populations (FIG. 3A). NMDA antagonists likewise inhibited the response to BzATP. Application of 10 μM MK-801 decreased the Ca²⁺ elevation by 50.1±5.5% when compared to mean levels immediately before and after (FIG. 3B). APV blocked the response by 36.1±7.5% (FIG. 3C) while memantine (FIG. 3D) inhibited it by 18.4±4.2%. The effect of NMDA antagonists on purified ganglion cells is summarized in FIG. 3E.

### Receptor Co-Localization

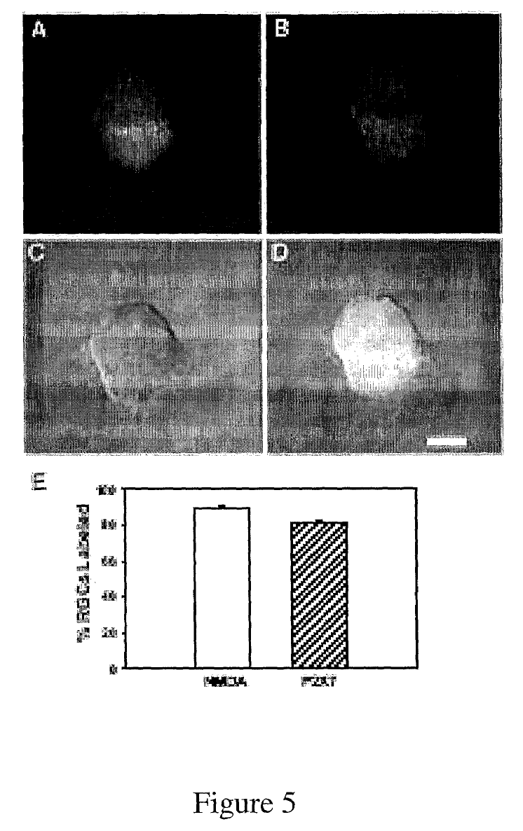

The ability of a P2X7R agonist and NMDAR antagonists to act on purified ganglion cells implied both receptors were present on retinal ganglion cells. This was confirmed immunohistochemically with >75% of neurons staining for both receptors (FIG. 5A). In total, 40 fields from 4 separate preparations were analyzed, with a mean cell number of 12.9±0.8 cells per field as determined with the nuclear stain DAPI. Initial trials indicated 89.3±1.7% of cells contained NMDA receptors, with 79.9±1.8% of cells stained for the P2X7 receptor raised against AAs 123-456 (n=30; FIG. 5B). As staining with this antibody was previously found to vary in some neural preparations, experiments were repeated with a second P2X7 antibody raised against AAs 987-654. A similar pattern of staining was found with 82.6±3.5% (n=10) of cells staining for P2X7 and 87.5±2.6% of cells staining for NMDAR Consistency between these two antibodies for the P2X7 receptor was previously taken to indicate specific staining {Sim, 2004 #1}, and suggested the antibodies were specific for the P2X7 receptor in retinal ganglion cells. In both cases, nearly all of the cells staining for the P2X7 receptor were positive for the NMDA receptor.

The staining for both NMDAR and P2X7R was predominately particulate, although distinct clusters were lost in the most densely stained regions. In these areas of intense staining, considerable overlap between the receptors was observed (FIG. 5D). However, distinct staining for only one receptor or the other was detected throughout the membrane.

### Mechanism of Receptor Interaction

The presence of both P2X7 and NMDA receptors on retinal ganglion cells, combined with the ability of NMDA antagonists to attenuate the rise in Ca2+ triggered by BzATP, suggested that activation of the P2X7 receptor led to a downstream activation of the NMDA receptor. In many neural systems, opening of channels precedes opening of the NMDA receptor. The membrane depolarization following activation of the initial receptor lessens the voltage-dependent block of the NMDA channel by Mg⁺⁺ and the channel opens. This mechanism is unlikely to account for the results above as the to measurements of intracellular Ca²⁺ were performed in the absence of extracellular Mg⁺⁺. However, receptor activation did require binding of the agonist glutamate. Therefore, the question of whether activation of the P2X7 receptor led to an increase in extracellular glutamate levels was investigated.

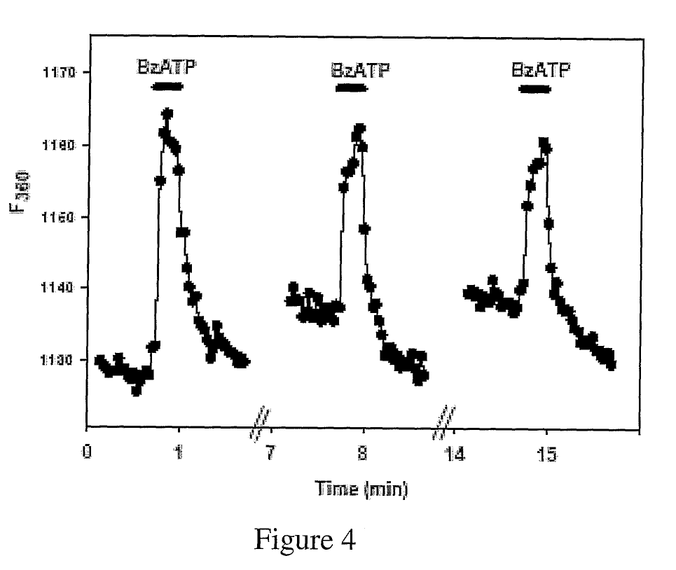

The extracellular glutamate levels bathing isolated ganglion cells were determined. Levels of glutamate in the bath remained stable while cells were perfused with a control solution, but the introduction of BzATP led to a large and rapid increase in glutamate levels (FIG. 4). Glutamate fell quickly after removal of BzATP, with levels returning close to baseline, consistent with the cessation of release in a perfused system. Subsequent application of BzATP led to additional rapid increases in extracellular glutamate levels that were usually similar in magnitude to the first. The pattern of glutamate release triggered by BzATP mirrored the pattern of Ca2+ elevation (FIG. 3A). In total, BzATP increased the absorbance at 360 nm by 17.1±4.0% in 17 applications from 7 coverslips. Each coverslip contained 6-50 ganglion cells in the field, and release was proportional to cell number. The response from individual cells was detectable at initial time points in coverslips with low numbers of cells. As the glutamate spread rapidly, levels were increased homogeneously across the field within 10 sec of BzATP application in coverslips with a moderate density of cells. The P2X7 antagonist brilliant blue G (BBG) eliminated the release of glutamate in response to BzATP, with a net absorbance change of −0.9±0.9% (n=3).

### Example 2

### NMDAR Kills Neurons Following P2X7R Activation

Stimulation of the P2X7 receptor leads to the activated of caspases and death of retinal ganglion cells. In light of the present findings demonstrating a role for glutamate in the large Ca²⁺infux following BzATP application, and as excess influx of Ca²⁺ through the NMDA receptor can lead to neuronal death, the activation of NMDA receptors contribution to the cell death accompanying BzATP was investigated.

Labeled ganglion cells in mixed retinal cultures were incubated under various conditions and the number of ganglion cells surviving after 24 hrs was determined. BzATP significantly reduced cell survival, with levels falling to only 62.9% of control. However, 10 μM MK-801 completely prevented the loss of cells, with levels rising to 102.7±3.2% of control. Morphologically, the surviving cells were indistinguishable from those under control conditions. Cell death was also reduced by APV, albeit to a smaller extent with survival levels increasing to 77.8±5.9%.

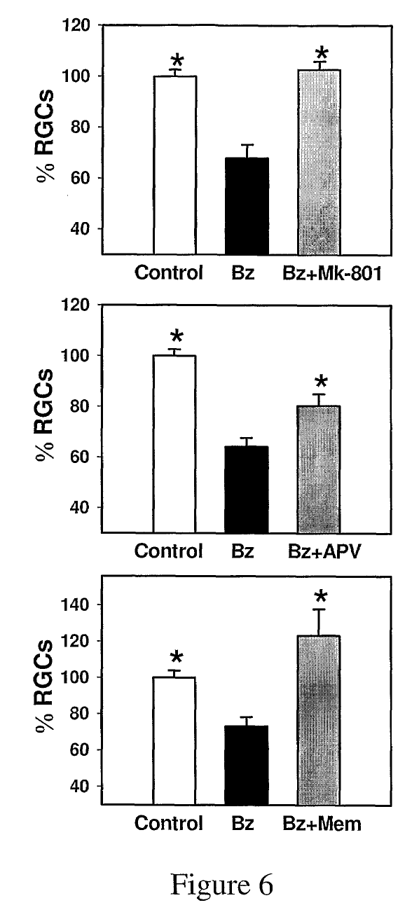

As shown in FIG. 6, NMDA antagonists reduce lethal effects of BzATP. (A) While incubation with 50 μM BzATP for 24 hrs reduced the number of surviving retinal ganglion cells compared to that in control solution, this loss was prevented by 10 μM MK-801 (*—diff from BzATP alone, Dunnett's test, N=9). (B) The antagonist APV (100 mM) also increased cell survival (*—diff from BzATP alone, Dunnett's test, N=15). (C) At 100 μM, memantine was also neuroprotective, raising the number of surviving cells considerably above that found in BzATP alone (*—diff from BzATP alone, Dunn's test on ranks due to enhanced variation, memantine not different from control, N=18).

### Example 3

### Adenosine Prevents the Rise in Ca²⁺ Triggered by BzATP

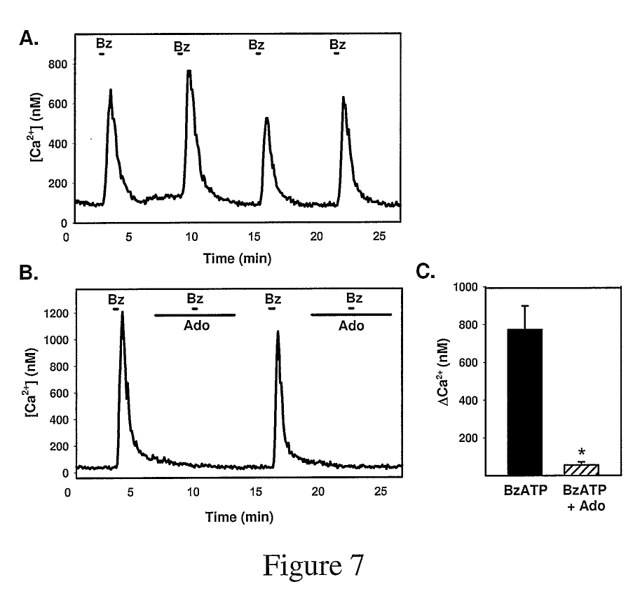

Stimulation of the P2X₇ receptor with agonist BzATP led to large elevations in cytoplasmic Ca2+. Sustained application of BzATP was previously shown to evoke a sustained increase in Ca²⁺ [see above examples]. However, brief application of 50 uM BzATP led to large and transient elevations in Ca²⁺ (FIG. 7A). With a periodic wash in between applications, repeated exposure to BzATP led to repeated elevations in Ca²⁺ that showed little evidence of a reduction in amplitude. This was previously shown to be due to the influx of Ca²⁺into the cell and involve the P2X₇ receptor [see above examples]. The ability to evoke recurring responses was used to examine the effect of adenosine on the Ca²⁺ response. Although BzATP was able to raise Ca²⁺ when applied alone, addition of adenosine greatly reduced the response (FIG. 7B). Removal of adenosine and subsequent reapplication of BzATP triggered another large elevation, indicating the block was reversible. A second application of adenosine inhibited the BzATP response, indicating the block was repeatable.

Lower concentrations of adenosine did produce a block, but the results were not consistent. As initial trials indicated that the block was not as effective if adenosine was given simultaneously with BzATP, a 3 min pretreatment was used in subsequent trials. Likewise, simultaneous removal of BzATP and adenosine occasionally led to a delayed rise in Ca²⁺; this delayed response was eliminated by extending the presence of adenosine for several min after BzATP removal. The effects of adenosine on extended applications of BzATP were examined. A two minute application of 50 uM BzATP raised peak Ca²⁺ levels to only 215±62 nM (n=7) in the presence of 300 uM adenosine (2 min pretreatment). This represents an 85% block as compared to the increase observed with BzATP alone and indicated the inhibition is sustained.

### Example 4

### Adenosine Protects from P2X7- and NMDA-Receptor Associated Death

BzATP led to the death of retinal ganglion cells when incubated with the cells for 4-48 hrs (see above examples). As adenosine block the Ca²⁺ rise triggered by brief and sustained applications of BzATP, and as excess Ca²⁺is toxic to many neurons, the ability of adenosine to prevent cell death was examined. Fluorescently labeled ganglion cells present in mixed retinal cultures plated on coverslips were exposed to adenosine for 30 min before addition of BzATP.

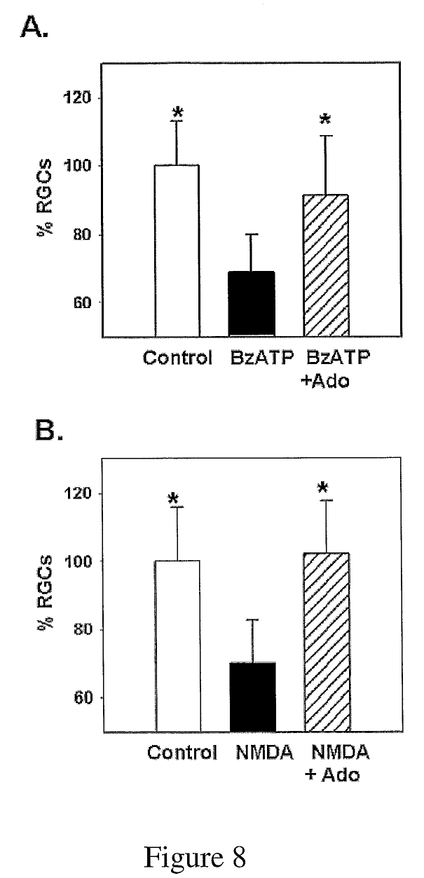

Adenosine protected ganglion cells from the cell death triggered by BzATP. While BzATP decreased the number of viable cells remaining after 24 hours to 68.9±2.3% of control, 300 uM adenosine increased cell survival to 91.2±3.5%. (FIG. 8A) Increasing the adenosine concentration to 1 mM produced similar results, increasing survival to 92.4±2.5% of control. The results produced by lower levels of adenosine were inconsistent in agreement with Ca²⁺ measurements.

Adenosine has been shown to inhibit Ca²⁺ elevations triggered by glutamate and glutamate agonist NMDA in rat retinal ganglion cells [see above examples]. As stimulation of the NMDA receptor is known to kill ganglion cells, the effect of adenosine on the lethal effects of NMDA was examined. At 100 uM, NMDA killed similar proportion of ganglion cells at 50 uM BzATP, with levels falling to 69.9±3.2% of control after 24 hrs (FIG. 8B). Exposure to 300 uM adenosine completely eliminated cell loss, increasing cell counts to 102.1±3.9% of control.

### Example 5

### A3 Receptor Contributes to Effects of Adenosine

Adenosine could have acted at A₁, A₂A, A₂B or A₃ receptors at the levels used to prevent Ca²⁺ elevations and cell death. Although the A₁ receptor is involved in attenuating the NMDA-triggered increase in Ca²⁺ [see above examples], both A1 and A3 adenosine receptors can be protective in neurons. As the A3 receptor was recently identified in retinal ganglion cells, the contribution of the A3 receptor to the Ca²⁺ block was examined pharmacologically.

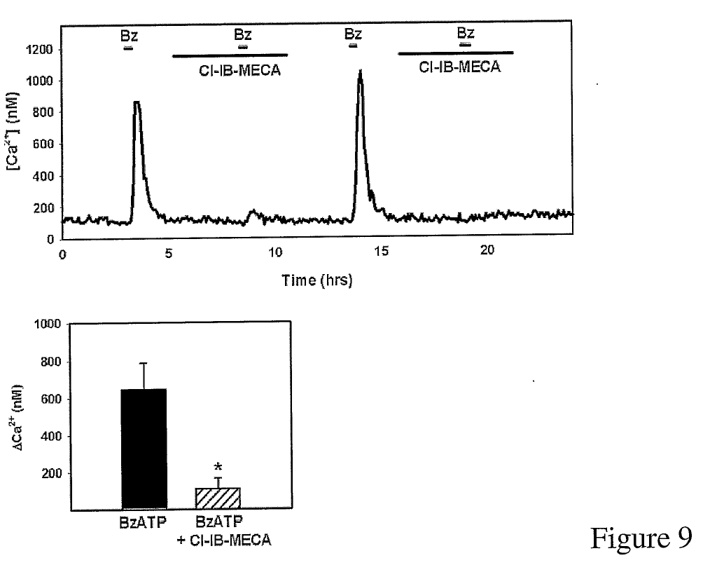

The agonist CI-IB-MECA shows considerable selectivity for the A3 receptor, with binding displacements of 820/420/0.33 nM at A1/A2A and A₃ receptors respectively, while the Kd for CI-IB-MECA at human A₂B receptors is >100,000. Employing a protocol analogous to that used above with adenosine, 100 nM CI-IB-MECA produced a reversible block of the Ca²⁺ rise induced by 15 sec exposure to BzATP (FIG. 9A). Quantification indicated the result was complable to that observed with adenosine. Mean levels of Ca²⁺ rose to 647.0±80.7 nM with the first exposure to BzATP, only 110.7±32.8 nM in the presence of CI-IB-MECA during the second BzATP application, but up to 600.3±227.6 nM for the third exposure after removal of CI-IB-MECA (FIG. 9B). When levels were normalized to the peak of the first response, CI-IB-MECA blocked the increase by 81%.

### Example 6

### Stimulation of the A3 Receptor is Neuroprotective

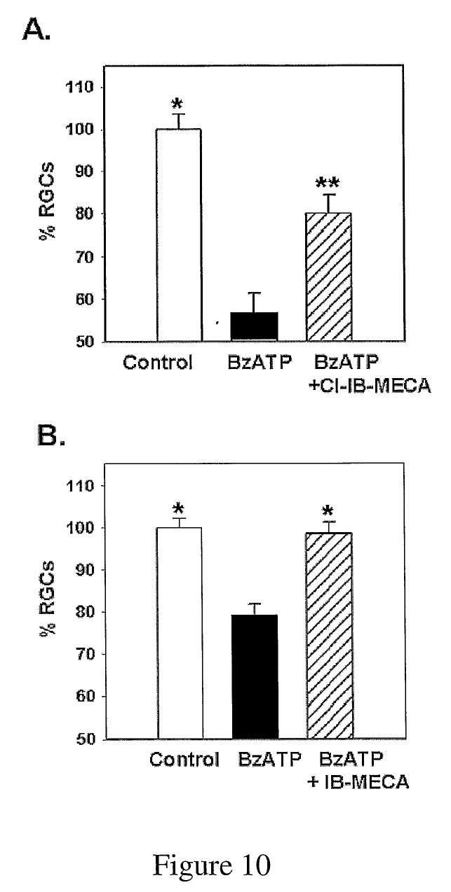

The effect of A3 agonists on cell viability was examined 24 hrs after addition of BzATP as above. In the presence of 50 uM BzATP the number of cells surviving was only 56.8% of control (FIG. 10A). However the proportion rose to 80.1±4.7% of control when 100 nM CI-IB MECA was included in the bath for 30 min before and during the BzATP. This represents a 54% reduction in the number of dead cells.

The neuroprotective contribution of the A3 receptor was further examined with the agonist IB-MECA. IB-MECA has binding displacements of 54/56/1 nM nM at A₁A₂A and A₃ receptors respectively. In this set of experiments, 50 uM BzATP reduced the number of viable cells to only 79.0±2.9% of control. However 100 nM IB-MECA increased survival to 98.5±2.7% of control. Using the same calculations as above, this indicated IB-MECA can protect against 95% of the cell death triggered by BzATP.

### Example 7

### Protection Conferred from Hydrolysis of ATP

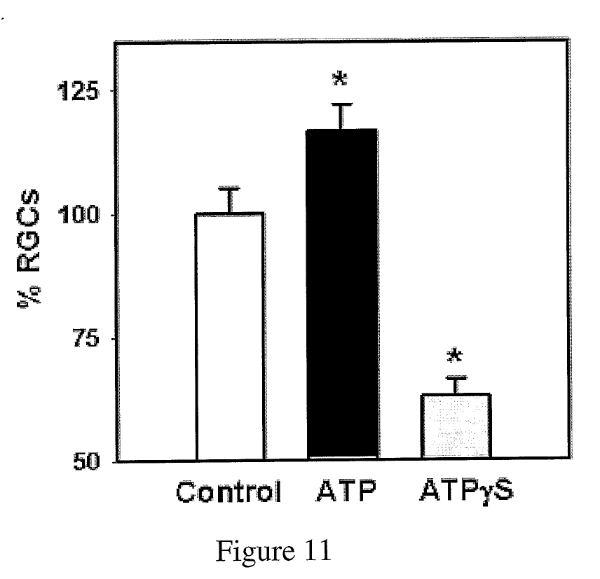

While BzATP is an effective pharmacologic tool at the P2X₇ receptor, the primary endogenous agonist is likely to be ATP. In contrast to the effect of BzATP, however, incubation of ganglion cells with 300 uM ATP for 24 hrs led to a small but significant increase in cell survival (FIG. 11A). The EC50 for ATP at the rat P2X₇ receptor is 300 uM, and measurements of ganglion cell Ca²⁺ levels indicate ATP can initiate a response over the short term [see above examples]. However, extracellular ATP is subject to rapid hydrolysis to adenosine by a variety of ecto-ATPases, and it was possible that the ATP was being dephosphorylated before it had sufficient time to stimulate the receptor. To test this hypothesis, the experiment was repeated with the ATP analog ATPyS, as the terminal phosphate is dephosphorylated at a much slower rate. When used at the same concentration, ATPyS reduced the cell number to 62.9±3.5% (FIG. 11B), similar to the reduction found with 50 uM BzATP.

### Example 8

### Adenosine Prevents Death of Retinal Ganglion Cells Following P2X7 Receptor Activation by Acting at A3 Receptors

The purines ATP and adenosine can work together as a coordinated team of transmitters. As extracellular adenosine frequently comes from the dephosphorylation of released ATP, the distinct actions of the two purines are synchronized. Stimulation of the P2X₇ receptor for ATP is known to produce excessive increases in intracellular Ca²⁺ and kill retinal ganglion cells. Here the effect of adenosine on this lethal action were examined. Adenosine attenuated the rise in Ca²⁺ produced by the P2X₇ agonist BzATP. Adenosine was neuroprotective, increasing survival of ganglion cells exposed to BzATP for 24 hrs. Adenosine also prevented cell death due to the glutamate agonist NMDA, suggesting the protection involved a common pathway. The A₃ adenosine receptor agonist CI-IB-MECA mimicked the inhibition of the Ca²⁺ rise. Both CI-IB-MECA and a second A₃ receptor agonist IB-MECA reduced cell loss triggered by BzATP. The actions of BzATP were mimicked by slowly-hydrolyzed ATPγS, but not ATP. In summary, adenosine can stop the rise in Ca²⁺ and cell death resulting from stimulation of the P2X₇ receptor on retinal ganglion cells, with the A₃ adenosine receptor contributing to protection. Hydrolysis of ATP into adenosine shifts the balance of purinergic action from cell death to cell preservation and suggests the ecto-enzymes responsible for this hydrolysis can be neuroprotective.

### Example 9

### Upregulation of Ecto-Nucleotidases Enhances Conversion of ATP to Adenosine

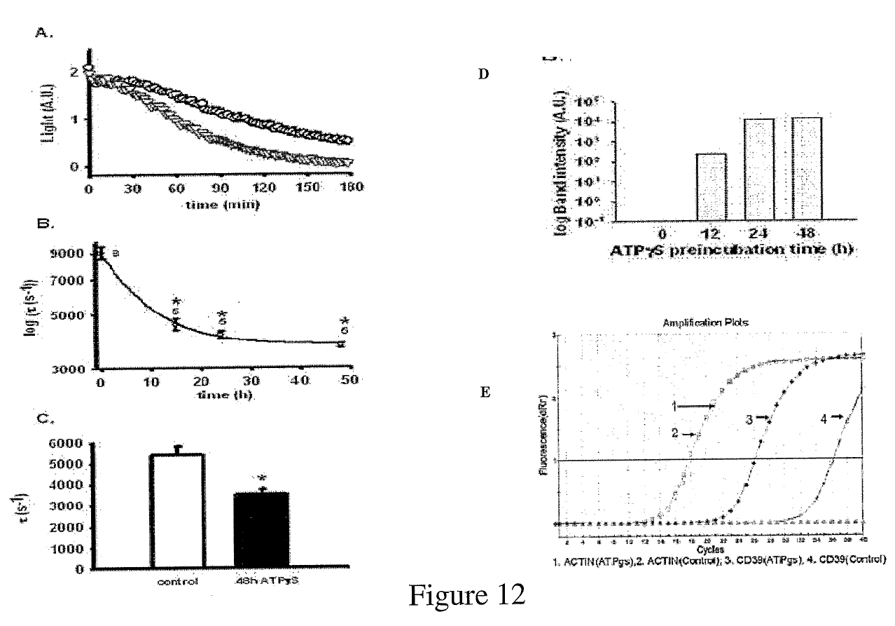

Stimulation of the P2X7 receptor for ATP cytotoxic and stimulation of the A3 receptor for adenosine neuroprotective, indicates that enhancing the conversion of ATP into adenosine is beneficial on two fronts. With regards to retinal pigmented epithelial cells, upregulating activity of the enzyme ecto-nucleoside triphosphate diphosphohydrolase (NTPDase)1 with purinergic agonist ATPγS was found to be possible through an increased transcription (see FIG. 12). As NTPDase 1 catalyzes the dual dephosphorylation of ATP to ADP and then to AMP, this upregulation protect ganglion cells in two ways. Appropriate purinergic agonists responsible for a parallel increase in retinal ganglion cells may be used to prevent cell death in glaucoma and other optic nerve neuropathies.

In ARPE 19 cells, preincubation with ATPγS produces an increase in ecto-ATPase activity. (A) 48 h preincubation with 100 μM ATPγS (grey triangles) produce an increase in the degradation ratio of 1 μM ATP added in the extracellular medium when is compared with non-preincubated controls (black circles). (B) All the ATP degradation records were fitted individually to exponential decay functions (y=ae^{(-bX)}) and the time constant (τ=1/b) was calculated at different preincubation times with ATPγS. The time constant of ATP degradation is reduced exponentially when the preincubation time with ATPγS is increased. (C) The time constant of degradation is significantly reduced after preincubation with ATPγS in 3 different experimental days totalizing 43 and 50 wells for control and 48 h preincubation time respectively. Symbols and bars represent the mean±SE. In FIG. 4A, error bars are smaller than symbols. * represents a significant difference with non-preincubated controls (ANOVA or Students t-test statistics, p<0.05). (D) Western blot analysis for NTPDase1 protein level from ARPE-19 cells. Incubation of cells with ATPγS led to an increase in 78 kDa bands on immunoblots after 12 hour, 24 hour and 48 hour preincubation in ARPE-19 cells. (E) Quantitative real-time PCR analysis of NTPDase1 expression in RPE cells. Amplification plots from a quantitative real-time PCR of NTPDase1 in the RPE cells exposed to normal or ATP□S medium was performed by SYBR Green real-time PCR. cDNA samples were diluted 1/10 and all reactions performed in triplicate. The Ct of house keeping gene β-actin similar in both normal and ATPγs medium (line1, 2), whereas Ct of NTPDase1 was much lower in ATPγs medium (line 3) compare with normal medium (line4)

### Example 10

### Increased Pressure Triggers ATP Release from the Retina

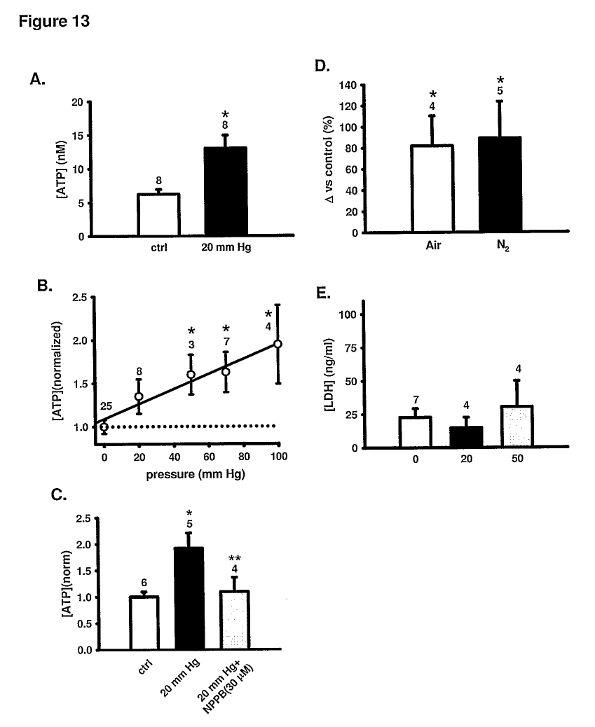

Many cells are known to release ATP in response to increased pressure. As stimulation of the P2X₇ receptor for ATP can kill ganglion cells, and as ganglion cells die in glaucoma frequently characterized by an increase in pressure, increased pressure could lead to ATP release in the posterior eye. Elevating pressure led to a clear release of ATP (see FIG. 13). This release was blocked by the Cl channel blocked NPPB, did not exhibit changes in partial pressure and did not exhibit cell damage. Therefore, elevated pressure triggers a release of ATP from retinal cells, which can lead to cell damage in glaucoma. Preventing this release with NPPB or other, more specific blockers may prevent ganglion cell death in glaucoma and other optic neuropathies.

As shown in FIG. 13, increase in extracellular pressure produces an increase in extracellular ATP concentration in the retina of bovine eyes.

A. A significant increase in the ATP concentration is detected in the sample obtained after the exposition of the retina eyecup to 20 mm Hg of extracellular pressure for 10 minutes (black bar) versus the non-pressured eyecup control samples (white bar).

B. The ATP concentration increases in the samples collected from the retina eyecup after 20 minutes of pressure challenge in a linear correlation with the increase of the amount of pressure between 20 and 100 mm Hg (r²=0.947). The bars and circles represent the mean±standard error. Numbers indicates the number of retina eyecups per experiment. We performed t-student test or ANOVA test (Tuckey post-test) to obtain the significant differences indicated by a * symbol (p<0.05).

C. ATP release by increased extracellular pressure is inhibited by the general chloride channel blocker NPPB, indicating physiologic significance. A 15 minutes preincubation with a 30 μM NPPB solution before the pressure experiment and the following addition of 30 μM NPPB in the extracellular medium (grey bar) inhibits the ATP release induced by a 20 mm Hg rise in extracellular pressure for 0.10 minutes (black bar). The ATP levels were normalized with the non-pressure and non NPPB eyecup control levels (white bar). Bars represent the mean±standard error. Numbers indicates the number of retina eyecups per experiment. We performed ANOVA test (Dunnett post-test) to obtain the significant differences indicated by a * symbol (p<0.05) as significant difference with control and ** (p<0.05) as significant difference with the pressure challenged eyecups.

D. The percentage of increase in ATP concentration in the pressured eyecup samples versus the non-pressure eyecup controls samples are not significant different if the increase in pressure is produced by introducing atmospheric air to the chamber (white bar) or by using pure nitrogen gas (black bar), indicating the release is due to elevated pressure and not changes in partial pressure.

E. LDH levels did not increase with pressure, indicating the release of ATP was physiologic and did not reflect cell damage. The extracellular LDH levels are not significant increased in samples from retina eyecup challenged with 20 (black bar) and 50 mm Hg (grey bar) for 10 minutes versus the control levels in samples collected from non-pressure eyecups (white bar). Bars represent the mean±standard error. Numbers indicates the number of retina eyecups per experiment. We performed t-student test or ANOVA test (Tukey post-test) to obtain the significant differences indicated by a * symbol (p<0.05).

### Example 11

### Chronic Glaucoma Results in Sustained Elevation in ATP

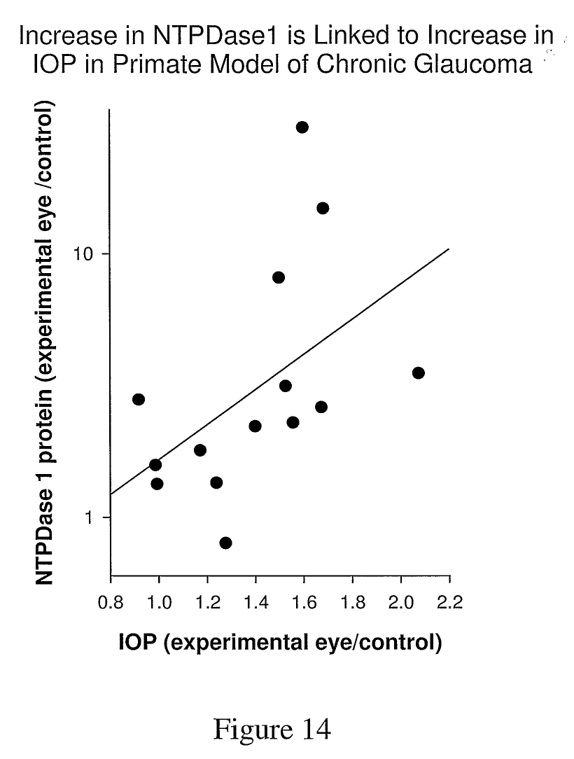

While the ability of a transient elevation in ocular pressure to trigger a release of ATP demonstrates a link between pressure and excess ATP, application to patients with chronic glaucoma requires demonstration that ATP is elevated over extended periods of time in primate models of chronic glaucoma. As was shown in the previous examples, the enzyme NTPDase1 acts as a marker for sustained levels of excess extracellular ATP. The levels of NTPDase1 in 15 pairs of primate eyes in which the intraocular pressure was increased in one eye was examined following laser coagulation of the trabecular meshwork. Protein levels were quantified using Western blots, with results typically repeated 3 times. NTPDase levels were higher in the treated eye in 14 out of 15 pairs. The relative increase in protein expression and the relative increase in pressure were also correlated (FIG. 14). These observations provide strong evidence for sustained elevations in ATP levels in primates with chronic glaucoma. They also show that the attempts to reduce stimulation the P2X₇ receptor described herein have direct advantage in the treatment of glaucoma patients, including chronic glaucoma.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A composition comprising at least two of a P2X₇ antagonist, a Ca²⁺ chelating agent, an NMDA receptor antagonist, Adenosine A3 receptor agonist or a combination thereof.

2. The composition of claim 1, further comprising a pharmaceutically acceptable carrier, excipient, flow agent, processing aid, a diluent or a combination thereof, thereby preventing the loss of cross-membrane potential and cell lysis.

3. The composition of claim 1, wherein said P2X₇ antagonist is calmidizolamide, OxATP, KN62, KN04, brilliant blue G, AZD9056, a hP2X₇-specific monoclonal antibody (MoAb) or a to combination thereof.

4. The composition of claim 1, wherein said NMDA receptor antagonist is memantine, 2-amino-5-phosphonovaleric acid (APV) or a combination thereof.

5. The composition of claim 1, wherein said adenosine A3 receptor agonist is adenosine (ADO), 2-chloro-N6-(3-iodobenzyl)-adenosine-5-N-methyluronamide (CI-IB-MECA), or a combination thereof.

6. The composition of claim 2, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

7. The composition of claim 1, comprising a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

8. The composition of claim 1, wherein said composition is in the form of a pellet, a tablet, a capsule, a solution, a suspension, a dispersion, an emulsion, an elixir, a gel, an ointment, a cream, or a suppository.

9. The composition of claim 1, wherein said composition is in a form suitable for oral, intravenous, intraaorterial, intramuscular, subcutaneous, parenteral, transmucosal, transdermal, or topical administration.

10. The composition of claim 1, wherein said composition is a liquid dosage form.

11. The composition of claim 1, wherein said composition is a solid dosage form.

12. A method for inhibiting or suppressing the reduction in number of retinal ganglion cells in a subject, comprising administering to said subject an effective amount of a composition comprising at least two of a P2X₇ antagonist, a Ca²⁺ chelating agent, an NMDA receptor antagonist , Adenosine A3 receptor agonist or a combination thereof, thereby preventing the stimulation of the receptor leading to death of ganglion cells and a reduction in their numbers.

13. The method of claim 12, wherein said P2X₇ antagonist is calmidizolamide, OxATP, KN62, KN04, brilliant blue G, AZD9056, or a combination thereof.

14. The method of claim 12, wherein said antagonist is a hP2X₇-specific MoAb

15. The method of claim 12, comprising removal of the P2X₇ agonist ATP, NAD⁺, mono-ADP-ribosyltransferases or their combination.

16. The method of claim 15, wherein the agonist is Ca²⁺ and removal is by administering a chelating agent.

17. The method of claim 12, wherein said subject exhibits increase in intraocular pressure.

18. The method of claim 17, comprising coadministering to said subject an effective amount of NMDA antagonist.

19. The method of claim 18, wherein said agonist is MK-801, 2-amino-5-phosphonovaleric acid (APV), memantine or a combination thereof.

20. The method of claim 12, further comprising activating the adenosine (ADO) A₃, A₁ receptors or their combination on said ganglion cells.

21. The method of claim 20, wherein said activation comprises contacting said ganglion cells with adenosine (ADO), 2-chloro-N6-(3-iodobenzyl)-adenosine-5-N-methyluronamide (CI-IB-MECA), or a combination thereof.

22. A method of treating a pathological condition in a subject resulting from a reduction in number of retinal ganglion cells, comprising administering to said subject a composition comprising at least two of a P2X₇ receptor antagonist, an adenosine A₃ receptor agonist, an adenosine A₁ receptor agonist, an agent capable of blocking the release of excessive ATP in response to elevated intraocular pressure, an ecto-nucleotidase agonist to convert extracellular ATP into adenosine, a Ca²⁺ chelating agent, an NMDA receptor antagonist, thereby reducing the stimulation of the P2X₇ receptors leading to death of ganglion cells, a reduction in their number thereby resulting in loss of function of said retinal ganglion cells.

23. The method of claim 22, wherein said pathological condition is glaucoma or ocular hypertension.

24. The method of claim 22, wherein said P2X₇ receptor antagonist is calmidizolamide, OxATP, KN62, KN04, brilliant blue G, AZD9056, or a combination thereof.

25. The method of claim 22, wherein said antagonist is a hP2X7-specific MoAb

26. The method of claim 22, wherein said subject exhibits increased, or erratic intraocular pressure, or their combination.

27. The method of claim 22, comprising removal of P2X₇ agonist.

28. The method of claim 27, wherein the agonist is ATP, NAD⁺, mono-ADP-ribosyltransferases or a combination thereof.

29. A method for the neuroprotection of the optic nerve in a subject, comprising administering to said subject an effective amount of an P2X₇ receptor antagonist, thereby preventing the stimulation of the receptor leading to death of retinal ganglion cells.

30. The method of claim 29, wherein said antagonist is calmidizolamide, OxATP, brilliant blue G, AZD9056, KN62, KN04 or a combination thereof.

31. The method of claim 29 , wherein said subject exhibits increase in intraocular pressure, or an erratic intraocular pressure, or their combination.

32. The method of claim 29, comprising removal of P2X₇ receptor agonist.

33. The method of claim 33, wherein the agonist is ATP, NAD⁺, mono-ADP-ribosyltransferases or a combination thereof.

34. A method for enhancing the conversion of ATP into adenosine in a retinal ganglion cell, comprising contacting said cell with an ecto-nucleotidase agonist and removing ATP thereby producing adenosine.

35. The method of claim 34, wherein increasing the activity of ectonucleotides comprises contacting the cell with a purinergic agonist, thereby upregulating expression of the gene encoding for ecto-nucleoside triphosphate diphosphohydrolase (NTPDase)1.

36. The method of claim 35, wherein said purinergic agonist is ATPγS or P2Y receptor agonists.

37. A method of reducing the release of cytotoxic ATP from a retinal cell in response to elevated intraocular pressure, comprising contacting said cell with an inhibitor of ATP release, thereby decreasing the release of excess ATP into the retina in response to elevated pressure.

38. The method of claim 37, wherein said inhibitor of ATP release is a Cl⁻ channel blocker, hemichannel blocker or a combination thereof.

39. The method of claim 38, wherein said Cl⁻ channel blocker is NPPB (5-nitro-2-(3-phenylpropyl-amino)benzoic acid), SITS (4-acetamido-4′-isothiocyanostilbene-2,2′-disulphonic acid), NFA (niflumic acid), DIDS (4,4′-diisothiocyanatostilbene-2,2′-disulfonic acid), A9C (anthracene-9-carboxylic acid), N-phenylanthranilic acid, DPC (diphenylamine-2-carboxylic acid), IAA-94 (R(+)methylindazone, indanyloxyacetic acid 94), 2-aminomethyl phenols, MK-447 (2-aminomethyl-4-(1,1-dimethyl ethyl)-6-iodophenol hydrochloride (2) disulfonic stilbenes, or a combination thereof.

40. The method of claim 38, where said hemichannel blocker is mefloquine acid, meclofenamic acid, retinoic acid, 18-α-glycyrrhetinic acid, flufenamic acid, niflumic acid, carbenoxolone, connexin mimetic peptides or a combination thereof.
